SEQUENCE LISTING (12 sequences)

SEQ ID NO: 1 - protein, 115 aa, artificial
  mature peptide of the amino acid sequence of the human VEGFC
AHYNTEILKSIDNEWRKTQCMPREVCIDVGKEFVATNTFFKPPCVSVYRCGGCCNSEGLQCMNTSTSYLSKTLFEITVPLSQGPKPVTISFANHTSCRCMSKLDVYRQVHSIIRR

SEQ ID NO: 2 - DNA, 358 bp, artificial
  nucleic sequence of the variable region heavy chain of the 1E9 antibodies
ccaggcccaactgcggcagcctggggctgaactggtgaagcctggggcttcagtgaagctgtcctgcaaggcttctggctacaccttcatcagctactggatgcactgggtgaaacagaggcctggacaaggccttgagtggattggagagattaatcctagcaacggtcgtactaactacaatgagaagttcaagagcaaggccacactgactgtagacagatcctccagcacagcctacatgcaactcagcagcctgacatctgaggactctgcggtctattactgtgcaagcacctctctcttttactatgctatgaattactggggtcaaggaacctcagtcaccgtctcctca

SEQ ID NO: 3 - DNA, 336 bp, artificial
  nucleic sequence of the variable region light chain  of the 1E9 antibodies
gatgttgtgatgacccaaactccactctccctgactgtcagtcttggagatcaagcctccatctcttgcagatctagtcagagccttgtacacagtaatggaaacacctatttacattggtacctgcagaagccaggccagtctccgaagctcctgatctacaaagttttcaaccgattgtctggggtcccagacaggttcagtggcagtggatcagggacagatttcacactcaagatcagtagagtggaggctgaggatctgggagtttatttctgctctcaaaatacgcatgttccgtggacgttcggtggaggcaccaagttggaaatcaaa

SEQ ID NO: 4 - protein, 119 aa, artificial
  amino acid sequence of the heavy chain variable region (VH)  of the 1E9 antibodies
QAQLRQPGAELVKPGASVKLSCKASGYTFISYWMHWVKQRPGQGLEWIGEINPSNGRTNYNEKFKSKATLTVDRSSSTAYMQLSSLTSEDSAVYYCASTSLFYYAMNYWGQGTSVTVSS

SEQ ID NO: 5 - protein, 112 aa, artificial
  amino acid sequence of the light chain variable region (VL)  of the 1E9 antibodies
DVVMTQTPLSLTVSLGDQASISCRSSQSLVHSNGNTYLHWYLQKPGQSPKLLIYKVFNRLSGVPDRFSGSGSGTDFTLKISRVEAEDLGVYFCSQNTHVPWTFGGGTKLEIK

SEQ ID NO: 6 - protein, 10 aa, artificial
  highly variable region CDR-H1 of the heavy chain of the 1E9 antibodies
GYTFISYWMH

SEQ ID NO: 7 - protein, 18 aa, artificial
  highly variable region CDR-H2 of the heavy chain of the 1E9 antibodies
GEINPSNGRTNYNEKFKS

SEQ ID NO: 8 - protein, 12 aa, artificial
  highly variable region CDR-H3 of the heavy chain of the 1E9 antibodies
ASTSLFYYAMNY

SEQ ID NO: 9 - protein, 16 aa, artificial
  highly variable region CDR-L1 of the light chain of the 1E9 antibodies
RSSQSLVHSNGNTYLH

SEQ ID NO: 10 - protein, 7 aa, artificial
  highly variable region CDR-L2 of the light chain of the 1E9 antibodies
KVFNRLS

SEQ ID NO: 11 - protein, 9 aa, artificial
  highly variable region CDR-L3 of the light chain of the 1E9 antibodies
SQNTHVPWT

SEQ ID NO: 12 - protein, 419 aa, homo sapiens
  amino-acid sequence of the human VEGFC
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMSL

(19) United States
(12) Patent Application Publication
(10) Pub. No.: US 2023/0192833 A1
(43) Pub. Date: Jun. 22, 2023

(54) New anti-VEGFC antibodies and uses thereof

(71) Applicants: CENTRE NATIONAL DE LA RECHERCHE (CNRS), PARIS (FR); INSERM (institut National de la Sante et de la Recherche Medicale), PARIS (FR); UNIVERSITE COTE D'AZUR, NICE (FR)

(72) Inventors: Gilles PAGES, MONACO (MC); Renaud GREPIN, BORDEAUX (FR); Aurore DUMOND, NICE (FR)

(21) Appl. No.: 17/906,627

(22) PCT Filed: Mar. 19, 2021
(86) PCT No.: PCT/EP2021/057070

§ 371 Date: Sep. 19, 2022

(30) Foreign Application Priority Data
Mar. 20, 2020 (EP) 20305296.4

(51) Int. Cl. C07K 16/22; C12N 15/63; A61P 35/04

(52) U.S. Cl. CPC C07K 16/22; C12N 15/63; A61P 35/04; C07K 2317/565; C07K 2317/24

(57) ABSTRACT

The invention relates to an isolated anti-vascular endothelial growth factor-C (VEGFC) antibody or a functional fragment thereof, said antibody comprising a heavy chain comprising at least one, preferentially at least two, preferentially three, of CDR-H1, CDR-H2 and CDR-H3 of amino acid sequences SEQ ID N° 6, 7 and 8 and a light chain comprising at least one, preferentially at least two, preferentially three of CDR-L1, CDR-L2 and CDR-L3 of amino acid sequences SEQ ID N° 9, 10 and 11, respectively.

### FIELD OF THE INVENTION

The present invention concerns new anti-VEGFC antibodies and their uses, in particular in the prevention and treatment of cancers and disorders characterized by undesirable lymphatic endothelial cell migration and/or proliferation.

### BACKGROUND OF THE INVENTION

In the last decades, angiogenesis, the formation of new blood vessels from a pre-existing vasculature, has emerged as a major target in age-related pathologies (including cancer, retinopathies, arthritis and rheumatoid arthritis). Several therapeutic agents targeting the VEGF-A165, the main pro-angiogenic factor and its associated receptors have been approved for cancer treatment (e.g. Bevacizumab (BVZ, Avastin®), a recombinant humanized monoclonal antibody, or sunitinib (Sutent®), a small-sized kinase inhibitor which targets specific VEGF receptors (Flt-1, Flk-1, Flt-4 as well as PDGF-R, CSFR1, e-KIT . . . ). For the patients, these conventional drugs lead to an indisputable initial period of clinical benefit, however they fail to definitively cure cancers. The treated primary tumors relapse and the remaining malignant cells disseminate to distant healthy tissues inducing metastases.

Therefore, new therapeutics are needed. This invention aims at developing a new strategy to tackle concomitantly the proangiogenic and the pro-lymphangiogenic pathways that are involved in tumor growth and metastatic dissemination.

To date, the mechanisms of tumor resistance are not fully understood [1]. In cancer, inflammation and angiogenesis are two closely integrated processes. Moreover, lymphangiogenesis is crucial since the lymphatic network is one of the main routes of metastatic dissemination. VEGFC is one of the major growth factors of lymphatic endothelial cells [2]. Several reports indicate that VEGFC expression in cancer cells correlates with accelerated tumor progression and a poor clinical outcome [3]. VEGFC overexpression in breast cancers has been shown to correlate with lymphangiogenesis and metastasis [4]. In preclinical models of RCC, endothelial cells chronically exposed to an anti-VEGF antibody proliferate in response to VEGFC stimulation whereas naive endothelial cells are unable to do so. In particular, it is demonstrated that VEGFC expression is induced following administration of bevacizumab or sunitinib in experimental models of (Renal Cell Carcinoma) RCC in mice [5, 6].

Renal cell carcinoma (RCC, also known as hypernephroma) is a renal cancer that originates in the lining of the proximal convoluted tubule, the very small tubes in the kidney that filter the blood and remove waste products. Clear cell metastatic renal cell carcinoma (ccRCC) is the most common type of kidney cancer in adults, responsible for approximately 80% of cases. Initial treatment is most commonly a radical or partial nephrectomy and remains the mainstay of curative treatment. Where the tumor is confined to the renal parenchyma, the 5-year survival rate is 60-70%, but this is lowered considerably where metastases have spread. It is resistant to radiation therapy and chemotherapy, but some cases respond to immunotherapy. Targeted cancer therapies such as sunitinib, temsirolimus, bevacizumab, interferon-alpha, and sorafenib have improved the outlook for RCC (progression-free survival), although they have not yet demonstrated improved survival.

Anti-angiogenic therapies have been used in clear cell renal cancers (ccRCC, the most common renal cancer) with mitigated results [7]. Although these treatments increase progression-free survival of patients, they do not impact the overall survival except in some extremely rare cases. One hypothesis that could explain this semi failure is the production by the tumor cells or cells of the tumor microenvironment of angiogenesis factors redundant to VEGF or its receptors (main therapeutic targets of current treatments).

The invention now provides new monoclonal antibodies directed against the part of VEGFC that specifically binds to and activates the VEGF receptor 3 (VEGF-R3), to specifically target the lymphatic pathway. In particular, the inventors demonstrated that the said anti-VEGFC antibodies inhibit the growth of experimental kidney tumors in nude mice. These antibodies inhibited the activation of VEGFR3 signaling and therefore the proliferation and the migration of VEGFC-stimulated endothelial cells. Moreover, they inhibited the proliferation of VEGFC-expressing renal cancer cells through NRP2 signaling. Whereas anti-VEGF antibodies have no effect or even favor experimental tumor growth in this model, the combination of an anti-VEGF and an anti-VEGFC have an additive effect on the inhibition of tumor growth. Therefore, these new antibodies may constitute a new therapeutic strategy for cancers and disorders characterized by undesirable lymphatic endothelial cell migration and/or proliferation, in particular a new therapeutic strategy for Renal cell carcinoma (RCC).

### SUMMARY OF THE INVENTION

A first object of the present invention is an isolated anti-vascular endothelial growth factor-C (VEGFC) antibody or a functional fragment thereof, said antibody comprising a heavy chain comprising at least one, preferentially at least two, preferentially three, of CDR-HI, CDR-H2 and CDR-H3 of amino acid sequences SEQ ID NO: 6, SEQ ID NO: 7 and SEQ ID NO: 8 and a light chain comprising at least one, preferentially at least two, preferentially three of CDR-L1, CDR-L2 and CDR-L3 of amino acid sequences SEQ ID NO: 9, SEQ ID NO: 10 and SEQ ID NO: 11, respectively.

Another object is a polynucleotide encoding a variable heavy chain (V_{H}) for an anti-VEGFC antibody according to the invention or a variable light chain (VL) for an anti-VEGFC antibody according to the invention.

The present invention also concerns an expression vector comprising the polynucleotide of the invention, a non-human host cell transformed with pairs of polynucleotides suitable for expressing an anti-VEGFC according to the invention, and a method of producing an anti-VEGFC antibody of the invention comprising:
culturing the non-human host cell, in particular non-human fibroblasts, under suitable conditions and
recovering the anti-VEGFC antibody from the culture medium or from the said cultured cells.

Another object of the present invention is a combination of an antibody anti-VEGFC according to the invention and an anti-angiogenic compound, in particular selected from the group consisting antibodies anti-VEGF distinct from the anti-VEGFC antibody of the invention, inhibitors of receptors involve in angiogenesis including VEGFR1, 2, 3, CSFR, PDGFR, inhibitors of m-TOR, preferably an anti-VEGF antibody distinct from the anti-VEGFC antibody of the invention.

The invention also concerns a pharmaceutical composition comprising an antibody according to the invention or a combination according to the invention and an excipient, carrier, and/or diluent.

The present invention also relates to an antibody as defined in the invention, or a combination of the invention or a pharmaceutical composition of the invention, for use as a medicament.

In a particular embodiment, the said antibody or the said combination or the said pharmaceutical composition are used in the treatment of cancers or disorders characterized by undesirable lymphatic endothelial cell migration and/or proliferation, in particular renal cancer carcinoma (RCC), breast cancer, head and neck cancers, retinopathies, arthritis or rheumatoid arthritis, preferably renal cancer carcinoma (RCC).

In a particular and preferred embodiment, the said antibody or the said combination or the said pharmaceutical composition are used in a subject who is relapsed from or refractory to its conventional treatment, and/or who is developing or is at risk of developing tumor metastasis.

Another object of the invention is an in vitro method for diagnosing or predicting the risk to relapse and/or to develop a tumor metastasis in a subject treated by its conventional treatment comprising an anti-angiogenic compound, characterized in that said method comprises a step of contacting a biological sample of said subject with an anti-VEGFC antibody of the invention, it being possible for said antibody to be, if necessary, labelled.

The present invention also relates to a kit for carrying out the in vitro method for diagnosing or predicting the risk to relapse and/or to develop a tumor metastasis in a subject treated by its conventional treatment comprising an anti-angiogenic compound, the said kit comprising at least one anti-VEGFC antibody and/or fragment thereof according to the invention and optionally at least one reagent for detecting said anti-VEGFC antibody.

### DETAILED DESCRIPTION OF THE INVENTION

### New Anti VEGFC Antibody or a Functional Fragment

The protein VEGFC (Vascular endothelial growth factor C) encoded by the human gene (Gene ID: 7424, RefSeq mRNA NM_005429) is a member of the platelet-derived growth factor/vascular endothelial growth factor (PDGF/VEGF) family. The encoded protein promotes endothelial cell proliferation and angiogenesis, and can also affect the permeability of blood vessels. It can also promote the proliferation of lymphatic endothelial cells and lymphangiogenesis. The proprotein is further cleaved into a fully processed form that can bind and activate VEGFR-2 and VEGFR-3 receptors.

VEGFC (Uniprot P49767, RefSeq protein NP_005420, amino-acid sequence represented by SEQ ID NO: 12) is proteolytically matured along a complex mechanism. The entire VEGFC molecule can stimulate both the VEGF receptor 2 (KDR) and VEGF receptor 3 (FLT 4) whereas the proteolytically cleaved form of VEGFC stimulates only VEGFR3 [8]. To specifically target the lymphatic pathway, the inventors immunized mice with the VEGFC form that only stimulates VEGFR3 according to the protocol described in FIG. 1. They obtained two monoclonal antibodies with a high specificity to VEGFC. The variable regions of these antibodies were sequenced (heavy and light chains). The CDR of these two antibodies were fused to human IgG1 light and heavy chains to obtain chimeric antibodies that can be used in the clinic. Expression vectors were transfected in CHO cells to obtain stable clones expressing the chimeric antibodies. The chimeric antibodies recognized with a high affinity the human VEGFC. As illustrated in the examples below, they inhibit the growth of experimental kidney tumors in nude mice. Whereas anti-VEGF antibodies have no effect or even favor experimental tumor growth in this model, the combination of anti VEGF and anti-VEGFC of the invention have an additive effect on the inhibition of tumor growth.

So a first object of the invention concerns an isolated anti-vascular endothelial growth factor-C (VEGFC) antibody or a functional fragment thereof, said antibody comprising a heavy chain comprising at least one, preferentially at least two, preferentially three, of CDR-H1, CDR-H2 and CDR-H3 of amino acid sequences SEQ ID N° 6, 7 and 8 and a light chain comprising at least one, preferentially at least two, preferentially three of CDR-L1, CDR-L2 and CDR-L3 of amino acid sequences SEQ ID N° 9, 10 and 11, respectively.

The antibody of the invention is obtainable by using an immunogen comprising a peptide antigen having an amino acid sequence corresponding to SEQ ID NO: 1.

In a particular embodiment, the antibody of the invention is able to compete with or inhibit the binding of VEGFC to VEGF-R3.

The CDR and framework regions in the murine sequences were identified using the IMGT-ONTOLOGY database [14, 15] and IMGT® databases and tools [16, 17]. More particularly, nucleotide and amino acid sequences of murine V_{H} and V_{L} domains were analyzed using IMGT/V-QUEST and IMGT/DomainGapAlign to delimit the murine CDRs and framework regions, define CDR lengths and identify anchor amino acids.

Unless defined otherwise, all technical and scientific terms used herein have the same meaning as commonly understood to one skilled in the relevant art.

For convenience, the meaning of certain term and phrases employed in the specification and claims are provided.

The antibodies described herein can be in the form of full-length antibodies, multiple chain or single chain antibodies, antigen binding fragments of such antibodies also named ‘functional fragments’, including but not limited to Fab, Fab′, (Fab′)₂, Fv, and scFv), single domain antibodies, humanized antibodies, camelid single-domain antibodies and the like. More particularly, an antibody (or “immunoglobulin”) consists of a glycoprotein comprising at least two heavy (H) chains and two light (L) chains inter-connected by disulfide bonds. Each heavy chain comprises a heavy chain variable region (or domain) (abbreviated herein as V_{H}) and a heavy chain constant region (hereafter C_{H}). Heavy chains are classified as gamma, mu, alpha, delta, or epsilon, and define the antibody's isotype as IgG, IgM, IgA, IgD, and IgE, respectively. The heavy chain constant region of the immunoglobulin IgG, IgD, and IgA (γ, δ and a chains respectively) comprises three domains (CH1, CH2, and CH3) and a hinge region for added flexibility, and the heavy chain constant region of the immunoglobulin IgM and IgE contains 4 domains (CH1, CH2, CH3, and CH4).

The antibody of the invention can be of the IgG, IgM, IgA, IgD, and IgE isotype, depending on the structure of its heavy chain. However, in a preferred embodiment, the antibody of the invention is of the IgG isotype, i.e., its heavy chain is of the gamma (γ) type.

IgG antibodies are classified in four distinct subtypes, named IgG1, IgG2, IgG3 and IgG4 in order of their abundance in serum (IgG1 being the most abundant). The structure of the hinge regions in the y chain gives each of these subtypes its unique biological profile (even though there is about 95% similarity between their Fc regions, the structure of the hinge regions is relatively different).

The antibody of the invention can be of the IgG1, IgG2, IgG3 or IgG4 subtype. However, in a preferred embodiment, the antibody of the invention is of the IgG1 subtype or of the IgG2 subtype, preferably of the IgG1 subtype.

Each light chain comprises a light chain variable region (abbreviated herein as V_{L}) and a light chain constant region comprising only one domain, C_{L}. There are two types of light chain in mammals: the kappa (κ) chain, encoded by the immunoglobulin kappa locus on chromosome 2, and the lambda (λ) chain, encoded by the immunoglobulin lambda locus on chromosome 22. In a preferred embodiment, the antibody of the invention has a Kappa light chain.

The V_{H} and V_{L} regions can be further subdivided into regions of hypervariability, termed “Complementarity Determining Regions” (CDR), which are primarily responsible for binding an epitope of an antigen, and which are interspersed with regions that are more conserved, termed “Framework Regions” (FR). Each V_{H} and V_{L} is composed of three CDRs and four FRs, arranged from amino-terminus to carboxy-terminus in the following order: FR1, CDR1, FR2, CDR2, FR3, CDR3, FR4. The assignment of amino acid sequences to each domain is in accordance with well-known conventions [9, 10]. The functional ability of the antibody to bind a particular antigen depends on the variable regions of each light/heavy chain pair and is largely determined by the CDRs. The variable region of the heavy chain differs in antibodies produced by different B cells but is the same for all antibodies produced by a single B cell or B cell clone (or hybridome).

By contrast, the constant regions of the antibodies mediate the binding of the immunoglobulin to host tissues or factors, including various cells of the immune system (e.g. effector cells) and the first component (Clq) of the classical complement system.

The term “antibody fragment” refers to a molecule comprising only a portion of an intact antibody, generally including an antigen binding site of the intact antibody and thus retaining the ability to bind antigen. Examples of antibody fragments encompassed by the present definition include: (i) the Fab fragment, having VL, CL, VH and CHI domains; (ii) the Fab′ fragment, which is a Fab fragment having one or more cysteine residues at the C-terminus of the CH 1 domain; (iii) the Fd fragment having VH and CH 1 domains; (iv) the Fd′ fragment having VH and CH 1 domains and one or more cysteine residues at the C-terminus of the CH 1 domain; (v) the Fv fragment having the VL and VH domains of a single arm of an antibody; (vi) the dAb fragment which consists of a VH domain; (vii) isolated complementarity determining regions (CDRs); (viii) F(ab′)2 fragments, a bivalent fragment including two Fab′ fragments linked by a disulphide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) “linear antibodies” comprising a pair of tandem Fd segments (VH-CH 1 -VH-CH 1) which, together with complementary light chain polypeptides, form a pair of antigen binding regions. Antibodies can be fragmented using conventional techniques. Various techniques have been developed for the production of antibody fragments. Traditionally, these fragments were derived via proteolytic digestion of intact antibodies. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

In particular, the isolated antibody or a fragment thereof, which is directed to the human proteolytically cleaved form of VEGFC, according to the invention comprises at least one, in particular at least two, at least three, at least four, at least five and more particularly six Complementary Determining Regions (CDRs) chosen among the CDRs of sequence comprising or consisting of SEQ ID No: 6, SEQ ID NO: 7, SEQ ID NO: 8, SEQ ID NO: 9, SEQ ID NO: 10, SEQ ID NO: 11, a sequence having at least 90%, at least 91%, at least 92%, at least 93%, at least 94%, at least 95%, at least 96%, at least 97%, at least 98%, at least 99% of identity with SEQ ID NO: 6 over the entire length of SEQ ID NO: 6, a sequence having at least 90%, at least 91%, at least 92%, at least 93%, at least 94%, at least 95%, at least 96%, at least 97%, at least 98%, at least 99% of identity with SEQ ID NO: 7 over the entire length of SEQ ID NO: 7, a sequence having at least 90%, at least 91%, at least 92%, at least 93%, at least 94%, at least 95%, at least 96%, at least 97%, at least 98%, at least 99% of identity with SEQ ID NO: 8 over the entire length of SEQ ID NO: 8, a sequence having at least 90%, at least 91%, at least 92%, at least 93%, at least 94%, at least 95%, at least 96%, at least 97%, at least 98%, at least 99% of identity with SEQ ID NO: 9 over the entire length of SEQ ID NO: 9, a sequence having at least 90%, at least 91%, at least 92%, at least 93%, at least 94%, at least 95%, at least 96%, at least 97%, at least 98%, at least 99% of identity with SEQ ID NO: 10 over the entire length of SEQ ID NO: 10, a sequence having at least 90%, at least 91%, at least 92%, at least 93%, at least 94%, at least 95%, at least 96%, at least 97%, at least 98%, at least 99% of identity with SEQ ID NO: 11 over the entire length of SEQ ID NO: 11, in particular chosen among the CDRs of sequence consisting of SEQ ID No: 6, SEQ ID NO: 7, SEQ ID NO: 8, SEQ ID NO: 9, SEQ ID NO: 10, or SEQ ID NO: 11.

The percentages of identity to which reference is made in the present invention are determined on the basis of a global alignment of sequences to be compared, that is to say, on an alignment of sequences over their entire length, using for example the algorithm of Needleman and Wunsch 1970. This sequence comparison can be done for example using the needle software by using the parameter “Gap open” equal to 10.0, the parameter “Gap Extend” equal to 0.5, and a matrix “BLOSUM 62”. Software such as needle is available on the website ebi.ac.uk worldwide, under the name “needle”.

In another embodiment of the antibody of the invention, the heavy chain variable region (VH) comprises:
a heavy chain CDR-H1 of sequence SEQ ID NO: 6 or of sequence having at least 90%, at least 91%, at least 92%, at least 93%, at least 94%, at least 95%, at least 96%, at least 97%, at least 98%, at least 99% of identity with SEQ ID NO: 6 over the entire length of SEQ ID NO: 6, in particular of sequence SEQ ID NO: 6; and/or
a heavy chain CDR-H2 of sequence SEQ ID NO: 7 or of sequence having at least 90%, at least 91%, at least 92%, at least 93%, at least 94%, at least 95%, at least 96%, at least 97%, at least 98%, at least 99% of identity with SEQ ID NO: 7 over the entire length of SEQ ID NO: 7, in particular of sequence SEQ ID NO: 7; and/or
a heavy chain CDR-H3 of sequence SEQ ID NO: 8 or of sequence having at least 90%, at least 91%, at least 92%, at least 93%, at least 94%, at least 95%, at least 96%, at least 97%, at least 98%, at least 99% of identity with SEQ ID NO: 8 over the entire length of SEQ ID NO: 8, in particular of sequence SEQ ID NO: 8; and
the light chain variable region (V_{L}) comprises:
a light chain CDR-L1 of sequence SEQ ID NO: 6 or of sequence having at least 90%, at least 91%, at least 92%, at least 93%, at least 94%, at least 95%, at least 96%, at least 97%, at least 98%, at least 99% of identity with SEQ ID NO: 9 over the entire length of SEQ ID NO: 9, in particular of sequence SEQ ID NO: 9; and/or
a light chain CDR-L2 of sequence SEQ ID NO: 10 or of sequence having at least 90%, at least 91%, at least 92%, at least 93%, at least 94%, at least 95%, at least 96%, at least 97%, at least 98%, at least 99% of identity with SEQ ID NO: 10 over the entire length of SEQ ID NO: 10, in particular of sequence SEQ ID NO: 10; and/or
a light chain CDR-L3 of sequence SEQ ID NO: 11 or of sequence having at least 90%, at least 91%, at least 92%, at least 93%, at least 94%, at least 95%, at least 96%, at least 97%, at least 98%, at least 99% of identity with SEQ ID NO: 11 over the entire length of SEQ ID NO: 11, in particular of sequence SEQ ID NO: 11.

In particular, the heavy chain variable region (VH) of the antibody according to the invention comprises:
a heavy chain CDR-H1 of sequence SEQ ID NO: 6; and
a heavy chain CDR-H2 of sequence SEQ ID NO: 7; and
a heavy chain CDR-H3 of sequence SEQ ID NO: 8

In particular, the light chain variable region (VL) of the antibody according to the invention comprises:
a light chain CDR-L1 of sequence SEQ ID NO: 9; and
a light chain CDR-L2 of sequence SEQ ID NO: 10; and
a light chain CDR-L3 of sequence SEQ ID NO:11.

In a particular embodiment, the heavy chain variable region (VH) comprises:
a heavy chain CDR-H1 of sequence SEQ ID NO: 6; and
a heavy chain CDR-H2 of sequence SEQ ID NO: 7; and
a heavy chain CDR-H3 of sequence SEQ ID NO: 8
and the light chain variable region (VL) comprises:
a light chain CDR-L1 of sequence SEQ ID NO: 9; and
a light chain CDR-L2 of sequence SEQ ID NO: 10 and
a light chain CDR-L3 of sequence SEQ ID NO:11.

In particular, the sequence of the heavy chain variable region (V_{H}) comprises or consists of the sequence SEQ ID NO: 4 or of a sequence having at least 90%, at least 91%, at least 92%, at least 93%, at least 94%, at least 95%, at least 96%, at least 97%, at least 98%, at least 99% of identity with SEQ ID NO: 4 over the entire length of SEQ ID NO: 4 and/or the sequence of the light chain variable region (V_{L}) comprises or consists of the sequence SEQ ID NO: 5 or of a sequence having at least 90%, at least 91%, at least 92%, at least 93%, at least 94%, at least 95%, at least 96%, at least 97%, at least 98%, at least 99% of identity with SEQ ID NO: 5 over the entire length of SEQ ID NO: 5.

In a particular and preferred embodiment, the sequence of the heavy chain variable region (VH) comprises or consists of the sequence SEQ ID NO: 4; and wherein the sequence of the light chain variable region (VL) comprises or consists of the sequence SEQ ID NO: 5.

The antibody of the invention can be a polyclonal or a monoclonal antibody, preferably a monoclonal antibody.

A “polyclonal antibody” as used herein, designates antibodies that are obtained from different B cell resources. It typically includes various antibodies directed against various determinants, or epitopes, of the target antigen(s). These antibodies may be produced in animals. Conventional techniques of molecular biology, microbiology and recombinant DNA techniques are within the skill of the art. Such techniques are explained fully in the literature. For example, the antibodies of the invention may be prepared by the following conventional method. A mammal (e.g. a mouse, rat, hamster, or rabbit) can be immunized with an immunogen comprising a peptide antigen having an amino acid sequence corresponding to SEQ ID NO: 1, which elicits an antibody response in the mammal. Techniques for conferring immunogenicity on a polypeptide include conjugation to carriers or other techniques well known in the art. For example, the polypeptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

In a preferred embodiment, the said antibody is a monoclonal antibody, in particular a mouse monoclonal antibody.

A “monoclonal antibody”, as used herein, means an antibody arising from a nearly homogeneous antibody population. More particularly, the subject antibodies of a population are identical except for a few possible naturally occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is generally characterized by heavy chains of one and only one isotype and subtype, and light chains of only one type. In addition, in contrast with preparations of polyclonal antibodies, each monoclonal antibody is directed against a single epitope of an antigen.

To produce monoclonal antibodies, antibody producing cells (lymphocytes) can be harvested from the immunized animal as described above and fused with myeloma cells by standard somatic cell fusion procedures thus immortalizing these cells and yielding hybridoma cells. Such techniques are well known in the art (e. g. the hybridoma technique originally developed by Kohler and Milstein (1975) as well as other techniques such as the human B-cell hybridoma technique [11], the EBV-hybridoma technique to produce human monoclonal antibodies [12], and screening of combinatorial antibody libraries [13]. Hybridoma cells can be screened immunochemically for production of antibodies specifically reactive with the target polypeptide(s) so that only monoclonal antibodies binding to said polypeptide(s) are isolated.

The antibody of the invention may be human, chimeric, humanized, murine, CDR-grafted, phage-displayed, bacteria-displayed, yeast-displayed, transgenic-mouse produced, mutagenized, and randomized.

In a particular and preferred embodiment, the antibody is a chimeric antibody.

A chimeric antibody is a molecule in which different portions are derived from different animal species, such as those having a variable region derived from a murine monoclonal antibody (mAb) and a human immunoglobulin constant region (See, e. g., Cabilly et al. (U.S. Pat. No. 4,816,567), and Boss et al. (U.S. Pat. No. 4,816,397)). Single-chain antibodies have an antigen binding site and consist of single polypeptides. They can be produced by techniques known in the art, for example using methods described in Ladner et al. (U.S. Pat. No. 4,946,778).

In another particular embodiment, the antibody is a humanized antibody.

Humanized forms of antibodies of the invention are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin (recipient antibody) are replaced by corresponding non-human residues of the donor antibody. Furthermore, humanized antibodies may comprise residues that are not found in the recipient antibody or in the donor antibody. In general, the humanized antibody may comprise substantially all of at least one, and typically two, variable domains, in which all or substantially all of the hypervariable loops correspond to those of a non-human immunoglobulin (donor antibody having the desired specificity, affinity, and capacity) and all or substantially all of the FRs are those of a human immunoglobulin sequence. In one embodiment, humanized antibodies comprise a humanized FR that exhibits at least 65% sequence identity with an acceptor (non-human) FR, e.g., murine FR. The humanized antibody also may comprise at least a portion of an immunoglobulin constant region (Fc), particularly a human immunoglobulin. Methods for humanizing non-human antibodies have been described in the art. Preferably, a humanized antibody has one or more amino acid residues introduced into it from a source, which is non-human. These non-human amino acid residues are often referred to as “import” residues, which are typically taken from an “import” variable domain. Humanization may be essentially performed by substituting hypervariable region sequences for the corresponding sequences of a human antibody. Accordingly, such humanized antibodies are chimeric wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. The choice of human variable domains, both light and heavy, to be used in making the humanized antibodies is very important to reduce antigenicity. Other methods generally involve conferring donor CDR binding affinity onto an antibody acceptor variable region framework. One method involves simultaneously grafting and optimizing the binding affinity of a variable region binding fragment. Another method relates to optimizing the binding affinity of an antibody variable region.

In a particular embodiment, for each of the murine monoclonal antibodies, corresponding humanized V_{H} and V_{L} peptide sequences were determined by: identifying the CDR and framework regions in the murine sequences using the IMGT-ONTOLOGY database [14, 15] and IMGT® databases and tools [16, 17] followed by identification of the amino acid sequence of the closest human framework region sequences in the IMGT/GENE-DB [18], and grafting of the murine CDR sequences onto the human framework regions. More particularly, nucleotide and amino acid sequences of murine VH and VL domains were analyzed using IMGTN-QUEST and IMGT/DomainGapAlign to delimit the murine CDRs and framework regions, define CDR lengths and identify anchor amino acids. Anchor amino acids are residues at position 26, 39, 55, 66, 104 and 118 of IMGT “Collier de Perles” that support the CDR1-IMGT, CDR2-IMGT, CDR3-IMGT [19, 20]. The closest human V (variable) and J (joining) genes to the murine sequences were identified and the most suitable genes chosen. Individual amino acids in the murine framework region were maintained if they were considered to possibly contribute to the specificity of the antibody by comparison with known 3D structures [21] using IMGT Collier de Perles on two layers.

Antibodies may be isolated after production (e.g., from the blood or serum of the animals) or synthetized and further purified by well-known techniques. Antibodies specific for a protein can be selected or purified by affinity chromatography, ELISPOT or ELISA. For example, the proteolytically cleaved form of VEGFC can be covalently or non-covalently coupled to a solid support such as, for example, a chromatography column. The column can then be used to purify antibodies directed to the proteolytically cleaved form of VEGFC, from a sample containing antibodies directed against a large number of different epitopes, thereby generating a substantially purified antibody composition, i.e., one that is substantially free of contaminating antibodies. By a “substantially purified antibody composition” it is meant, in this context, that the antibody sample contains at most only 30% (by dry weight) of contaminating antibodies directed against epitopes other than those of the Strep-Tag II sequence, and preferably at most 20%, yet more preferably at most 10% and most preferably at most 5% (by dry weight) of the sample is contaminating antibodies. A “purified antibody composition” means that at least 99% of the antibodies in the composition are directed to the proteolytically cleaved form of VEGFC.

The antibodies of the invention may be administered in their “naked” or unconjugated form or may have other agents conjugated to them. For examples the antibodies may be in detectably labelled form. Antibodies can be detectably labelled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, and the like. Procedures for accomplishing such labelling are well known in the art.

In another embodiment, the antibody of the invention is lyophilized. In such embodiment, the lyophilized antibody is admixed with a carrier or diluent such as those hereinabove described at the time of administration. In yet another embodiment, the antibody of the invention is conjugated to a compound such as a polymer. In one embodiment, the polymer is a polyalkylene glycol. In one embodiment, the polyalkylene glycol is polyethylene glycol, or PEG.

For use in therapy, the antibody(ies) will be suitably formulated together with pharmaceutically acceptable excipients. Suitable formulations for the preventive or therapeutical treatment can be administered parenterally, preferably intra-arterially, intraperitoneally, intravenously, subcutaneously, intramuscularly or via an aerosol, and they will contain an effective amount of the antibody(ies). Said amount will vary depending on the general conditions of the patient, the progression of the disease and other factors.

Production of the Said Antibody

The antibody of the invention can be obtained by immunizing animals, like rats or mice, with peptides specific for human proteolytically cleaved form of VEGFC, particularly with the mature peptide of sequence AHYNTEILKSIDNEWRKTQCMPREVCIDVGKEFGVATNTFFKPPCVSVYRCGGCCNSEG LQCMNTSTSYLSKTLFEITVPLSQGPKPVTISFANHTSCRCMSKLDVYRQVHSIIRR (SEQ ID NO: 1) of the VEGFC human protein ((NCBI Reference Sequence NP_005420), following by screening methods to isolate antibody. Different screening methods to isolate antibody can be used including ELISA tests on said peptide.

In a particular embodiment, the present invention also relates to an antibody obtained by, or susceptible to be obtained by, a method comprising the following steps:
i) immunizing at least an animal with at least one mature peptide of sequence AHYNTEILKSIDNEWRKTQCMPREVCIDVGKEFGVATNTFFKPPCVSVYRC GGCCNSEGLQCMNTSTSYLSKTLFEITVPLSQGPKPVTISFANHTSCRCMS KLDVYRQVHSIIRR (SEQ ID NO: 1) of the VEGFC human protein ((NCBI Reference Sequence NP_005420),
ii) generating monoclonal antibodies by any method known by a man skilled in the art, such as isolating B cells from said animal and fusing said B cells with myeloma cells to form immortal hybridoma cells that are able to secrete monoclonal antibodies,
iii) performing screening methods to isolate an antibody able to specifically bind to proteolytically cleaved form of VEGFC.

The invention also relates to a method for preparing an antibody directed to proteolytically cleaved form of VEGFC, comprising the steps of:
i) immunising a non-human animal by repeated administration of at least one peptide chosen in the group consisting of:

a peptide of sequence comprising or consisting of the sequence AHYNTEILKSIDNEWRKTQCMPREVCIDVGKEFGVATNTFFKPPCVSVYRCGGCCNSEG LQCMNTSTSYLSKTLFEITVPLSQGPKPVTISFANHTSCRCMSKLDVYRQVHSIIRR (SEQ ID NO: 1) and a peptide of sequence comprising or consisting of a sequence having at least 85%, at least 90%, at least 95% of identity with SEQ ID NO: 1 over the entire length of SEQ ID NO: 1;

and/or of at least one expression vector comprising a nucleotide sequence encoding said at least one peptide under the control of a promoter, which is effective in cells of said non-human animal; and
ii) collecting the resulting serum from said immunized non-human animal to obtain antibodies directed against said at least one peptide;
iii) determining in vitro or ex vivo the ability of said antibodies obtained at said step ii) to specifically bind to proteolytically cleaved form of VEGFC; and eventually
iv) selecting antibody(ies), which is (are) able to specifically bind to said proteolytically cleaved form of VEGFC.

Suitable non-human animals include but are not limited to mouse, rat, sheep, goat, hamster, rabbit, preferably mouse.

The peptide used as immunogenic peptide in step i) may comprise the complete peptide, or fragments and derivatives thereof, which are able to elicit a humoral immune response directed against the proteolytically cleaved form of VEGFC.

The serum of said animal can be sampled for evaluation of antibody titre. When the optimal titre has been reached, the animal can be bled to yield a suitable volume of specific serum. The degree of antibody purification required depends on the intended application. For certain purposes, there is no requirement at all for purification, however, in other cases, such as where the antibody is to be immobilised on a solid support, purification steps can be taken to remove undesired material and reduce or eliminate nonspecific binding.

Step iii) of determining in vitro or ex vivo the ability of said antibodies obtained at said step ii) to specifically bind proteolytically cleaved form of VEGFC, can be readily determined by one skilled in the art, for example, by Scatchard analysis (1949) or surface Plasmon resonance analysis as described hereafter.

The term “a nucleotide sequence encoding said at least one peptide under the control of a promoter, which is effective in cells of said non-human animal” relates to a nucleotide sequence encoding said at least one peptide, which is operatively linked to a promoter, which directs the expression of said nucleotide sequence in cells of said non-human animal.

In general, the vectors useful in the invention include but are not limited to plasmids, phagemids, viruses, other vehicles derived from viral or bacterial sources, which have been manipulated by the insertion or incorporation of a nucleotide sequence encoding said at least one peptide.

The expression of said at least one peptide can be stable or transitory expression, using stable or transitory expression vectors, respectively. Example of transitory expression vectors include, plasmids. Examples of stable expression vectors include lentiviruses vectors. Preferably, the expression of said at least one peptide is stable.

The expression of said at least one peptide can also be constitutive or inducible, using constitutive or inducible promoters, which are well known by one skilled in the art. Examples of constitutive promoters include mammalian or viral promoters like beta-actin promoter, muscle creatine kinase promoter, human elongation factor, promoters from the simian virus (e.g., SV40), papilloma virus, adenovirus, human immunodeficiency virus (HIV), cytomegalovirus (CMV), Rous sarcoma virus (RSV), hepatitis B virus (HBV), the long terminal repeats (LTR) of Moloney leukemia virus and other retroviruses, and the thymidine kinase promoter of herpes simplex virus. One can readily use other constitutive promoters not named but known in the art.

The promoters useful for the invention also include inducible promoters, which are expressed in the presence of an inducing agent. For example, the metallothionein promoter is induced to promote transcription and translation in the presence of certain metal ions. Preferably, the expression of said at least one peptide is constitutive.

In particular, the antibody of the invention is produced by the hybridoma.

The hybridoma has been obtained by immunization of mice with a peptide specific for human proteolytically cleaved form of VEGFC i.e. the peptide of sequence AHYNTEILKSIDNEWRKTQCMPREVCIDVGKEFGVATNTFFKPPCVSVYRCGGCCNSEG LQCMNTSTSYLSKTLFEITVPLSQGPKPVTISFANHTSCRCMSKLDVYRQVHSIIRR (SEQ ID NO: 1) of the VEGFC human protein ((NCBI Reference Sequence NP_005420).

This strategy was developed to obtain specific hybridoma producing anti-VEGFC antibodies. The screening was based on the ability of the hybridoma to recognize the VEGFC antigen used in the immunization protocol by ELISA. The flow-chart of the experiment is described in FIG. 2. Thirteen clones were selected for their ability to produce specific antibodies and the six best were amplified.

This hybridoma produces high yields of antibodies of the invention.

The antibodies according to the invention have in particular the following advantages:
They are unique described antibodies directed to proteolytically cleaved form of VEGFC;
Compared to Bevacizumab (antibody directed to VEGF) which increases tumor growth in experimental models [7], the antibodies of the invention inhibit tumors growth;
The antibodies of the invention target specifically the lymphatic pathway different of the classical angiogenic pathway. Hence by inhibiting lymphatic pathway, proliferation and/or migration of tumor cells (metastases) are prevented or inhibited by the antibodies of the invention.

The present invention also relates to a polynucleotide encoding a variable heavy chain (V_{H}) for an anti-VEGFC antibody according to the invention or a variable light chain (VL) for an anti-VEGFC antibody according to the invention.

The term “polynucleotide” refers to a polymeric form of nucleotides, either deoxyribonucleotides or ribonucleotides, or analogues thereof.

Another object of the invention is an expression vector comprising the said polynucleotide encoding an antibody or a fragment thereof according to the invention.

The vectors can be viral vectors such as bacteriophages or non-viral such as plasmids.

The present invention also concerns a non-human host cell transformed with pairs of polynucleotides suitable for expressing an anti-VEGFC according to the invention.

The invention also relates to a host cell comprising the polynucleotide encoding an antibody or a fragment thereof according to the invention or a vector comprising said polynucleotide.

Another object of the invention is a method of producing an anti-VEGFC antibody according to the invention comprising:
culturing the non-human host cell of the invention, in particular non-human fibroblasts, under suitable conditions and
recovering the anti-VEGFC antibody from the culture medium or from the said cultured cells.

Combination

Another object of the invention is a combination of an antibody anti-VEGFC according to the invention and an anti-angiogenic compound.

By “combination”, it refers to simultaneous (concurrent) or consecutive administration in any order. The combined administration includes co-administration, using separate formulations or a single pharmaceutical formulation or composition, and consecutive administration in any order. Thanks to the present invention, the subject receiving the combined treatment will be completely treated (cured), i.e., the subject will survive to the cancer [beneficial impact on the “overall survival” (OS)], or will have a much longer disease-free survival (DFS) or metastasis free survival chance than a subject who does not receive said combined treatment.

As used herein, the term “anti-angiogenic compound” refers to any compound that inhibits the development of a pathological vascular network. In particular, the anti-angiogenic compound can inhibit receptors of pro-angiogenic factors like VEGF receptors and receptors of CXCL/ELR+ chemokines (e.g., CXCR1 and CXCR2).

In particular, the anti-angiogenic compound is selected from the group consisting of:
Antibodies anti-VEGF, like bevacizumab, aflibercept (particularly for the treatment and/or prevention of cancers, in particular colorectal cancer);
Inhibitors of receptors involved in angiogenesis including VEGFR1, 2, 3, CSFR, PDGFR, like sunitinib, sorafenib, axitinib, cabozantinib, pazopanib, lenvatinib, regorafenib;
Inhibitors of m-TOR, like everolimus, temsirolimus;
And mixtures thereof.

In a particular embodiment, the anti-angiogenic compound is selected from the group consisting of antibodies anti-VEGF distinct from the anti-VEGFC antibody of the invention, inhibitors of receptors involve in angiogenesis including VEGFR1, 2, 3, CSFR, PDGFR, inhibitors of m-TOR, preferably an anti-VEGF antibody distinct from the anti-VEGFC antibody of the invention.

In a particular embodiment, the anti-angiogenic compound is an anti-VEGF antibody distinct from the anti-VEGFC antibody of the invention.

In a particular embodiment, the anti-VEGF antibody distinct from the anti-VEGFC antibody of the invention is selected from the group consisting of bevacizumab and aflibercept.

In a particular embodiment, the combination may further comprise another anti-angiogenic compound selected from inhibitors of receptors involved in angiogenesis including VEGFR1, 2, 3, CSFR, PDGFR, in particular selected from the group consisting of sunitinib, sorafenib, axitinib, carbozantinib, pazopanib, lenvatinib, and regorafenib.

The combination may also comprise at least another compound of interest (e.g. an anti-tumor agent, an anti-inflammatory compound).

As used herein, the term “anti-tumor agent” refers to any compound that prevents tumor growth or promotes tumor shrinking. Anti-tumor agents can be anti-angiogenic compounds, DNA intercalators/Cross-linkers (like oxaliplatin, mitoxantrone), DNA synthesis inhibitors (like cytosine β-D-arabinofuranoside, 5-Fluorouracil), DNA-RNA Transcription Regulators (doxorubicin, actinomycin D), Microtubule Inhibitors (paclitaxel, nocodazole).

As used herein, the term “anti-inflammatory compound” refers to any compound that reduces inflammation. In particular, anti-inflammatory compound can be corticoid and non-steroid anti-inflammatory agent ibuprofen derivative.

So the invention also relates to a combination product, which comprises:
an anti-VEGFC antibody or a fragment thereof according to the invention;
an anti-angiogenic compound; and
optionally another compound selected from anti-tumor compound and anti-inflammatory compound

for simultaneous, separate or sequential use as a medicament.

Pharmaceutical Composition

Another object of the invention is a pharmaceutical composition comprising an anti-VEGFC antibody according to the invention or a combination according to the invention and an excipient, carrier, and/or diluent.

Such antibody or combination can be present in the pharmaceutical composition or medicament according to the invention in a therapeutically effective amount (active and non-toxic amount). A therapeutically effective amount refers to that amount of compound which ameliorates the symptoms or condition. Therapeutic efficacy and toxicity may be determined by standard pharmaceutical procedures in cell cultures or experimental animals, e.g., ED50 (the amount therapeutically effective in 50% of the population) and LD50 (the dose lethal to 50% of the population). The amount ratio of toxic to therapeutic effects is the therapeutic index and it can be expressed as the ratio, LD50/ED50. Pharmaceutical compositions which exhibit large therapeutic indices are preferred.

For example, the antibody according to the invention, can be administered to a patient, in particular intravenously, in an amount, within the range from 0.1 μg/kg to 100 mg/kg, particularly from 1 mg/kg to 50 mg/kg of body weight of said patient at least every month, particularly at least every three weeks, more particularly at least every two weeks.

In the context of the invention, the anti-VEGFC antibody, or functional fragment thereof, is administered to the subject in a therapeutically effective amount [amount effective to reduce the number of cancer cells, kill cancer cells, reduce the tumor volume/size, or inhibit tumor growth, in particular when the anti-VEGFC antibody is used in combination with the conventional treatment] or prophylactically effective amount [amount effective to reduce or suppress lymphatic vessels formation or development and/or reduce or prevent the occurrence of new metastasis(es) or the development of existing metastasis(es)], typically at a concentration range from about 2 to 20 mg/kg of body weight, preferably from about 5 to 15 mg/kg of body weight, for example 6, 7, 8, 9, 10, 11, 12, 13 or 14 mg/kg of body weight, preferably 10 mg/kg of body weight.

The pharmaceutical composition according to the invention may be administered by any number of routes including, but not limited to, oral, intravenous, intramuscular, intraarterial, intramedullary, intrathecal, intraventricular, transdermal, subcutaneous, intraperitoneal, intranasal, enteral, topical, sublingual, rectal means or ocular.

In addition to the active ingredients, the pharmaceutical composition of the invention may contain suitable pharmaceutically acceptable carriers comprising excipients and auxiliaries, which facilitate processing of the active compounds into preparations which can be used pharmaceutically. In particular, the pharmaceutical composition according to the invention is formulated in a pharmaceutical acceptable carrier. Pharmaceutical acceptable carriers are well known by one skilled in the art. Further details on techniques for formulation and administration may be found in the latest edition of Remington's Pharmaceutical Sciences (Maack Publishing Co., Easton, Pa.). In particular, the pharmaceutical acceptable carrier comprises or is an isotonic solution.

A further object herein described is a kit or composition, comprising (i) a conventional treatment, and (ii) an anti-VEGFC antibody, or functional fragment thereof as herein described.

A “conventional treatment of cancer” may be selected from a chemotherapy, a radiotherapy, an hormonotherapy, an immunotherapy, a specific kinase inhibitor-based therapy, an antiangiogenic compound based-therapy, an antibody-based therapy, in particular a monoclonal antibody-based therapy, and surgery.

In particular, the conventional treatment comprises an anti-angiogenic compound. Such anti-angiogenic compounds are disclosed above.

Uses

Therapeutic Use

The present invention also relates to an antibody as defined in the invention, or a combination of the invention or a pharmaceutical composition of the invention, for use as a medicament.

In the context of the present invention, the patient or subject is a mammal. The mammal may be a primate, particularly a human being (also herein identified as “human”).

In a preferred embodiment, the mammal is a human being, whatever its age or sex.

In a particular embodiment, the subject has a tumor or cancer, typically a malignant tumor, in particular a metastatic (malignant) tumor.

In the context of the present invention, the cancer or “malignant tumor” may be any kind of cancer or neoplasia, typically metastatic cancer or neoplasia. The cancer or tumor can be selected from a carcinoma, a sarcoma, and a. The cancer is preferably selected from a renal or kidney cancer, preferably a renal cancer carcinoma (RCC), in particular a clear cell renal cancer carcinoma (ccRCC); a breast cancer; ovarian cancers, lung cancers, pancreatic cancers and colon cancers. In a particular embodiment, the cancer is a renal cancer carcinoma (RCC), in particular a clear cell renal cancer carcinoma (ccRCC).

In a particular embodiment, the said antibody or the said combination or the said pharmaceutical composition are used in the treatment of cancers or disorders characterized by undesirable lymphatic endothelial cell migration and/or proliferation, in particular renal cancer carcinoma (RCC), breast cancer, head and neck cancers, retinopathies, arthritis or rheumatoid arthritis, preferably renal cancer carcinoma (RCC).

The terms “treat”, “treating” or “treatment” refer to both therapeutic treatment and prophylactic or preventative measures, wherein the aim is to prevent or ameliorate cancer or prevent or slow down cancer progression or metastases appearance, development or multiplication. Those in need of treatment include those already with the disorder as well as those in which the disorder is to be prevented.

The terms “preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

By ‘disorders characterized by undesirable lymphatic endothelial cell migration and/or proliferation’, it means in particular disorders selected from the group consisting of:
cancers with abnormal angiogenesis, in particular solid tumors with abnormal angiogenesis, more particularly renal cancers including clear cell renal cell carcinoma, breast cancers, head and neck cancers, ovarian cancers, lung cancers, pancreatic cancers and colon cancers;
ophthalmological diseases with abnormal angiogenesis, in particular age-related macular degeneration, diabetic retinopathy, retinitis pigmentosa and uveitis;
rheumatoid arthritis;

Particularly, said pathological angiogenesis disease is a cancer, and preferably a clear cell renal cell carcinoma.

In a particular and preferred embodiment, the said antibody or the said combination or the said pharmaceutical composition are used in a subject who is relapsed from or refractory to its conventional treatment, and/or who is developing or is at risk of developing tumor metastasis.

In a particular embodiment of the present invention, the subject is typically a subject undergoing a treatment of cancer, in particular a conventional treatment of cancer (for example chemotherapy and/or radiotherapy). The term “conventionally” means that the therapy is applied or, if not routinely applied, is appropriate and at least recommended by health authorities. The “conventional” treatment is selected by the cancerologist depending on the specific cancer to be prevented or treated.

In the context of the present invention, a “conventional treatment of cancer” may be selected from a chemotherapy, a radiotherapy, an hormonotherapy, an immunotherapy, a specific kinase inhibitor-based therapy, an antiangiogenic compound based-therapy, an antibody-based therapy, in particular a monoclonal antibody-based therapy, and surgery.

The invention also relates to a method of treatment of diseases characterized by undesirable lymphatic endothelial cell migration and/or proliferation, said method comprises the step of administering to a patient in need thereof a therapeutically or prophylactic amount of at least one anti-VEGFC antibody or a fragment thereof according to the invention, a combination according to the invention or a pharmaceutical composition according to the invention, as disclosed above.

Diagnosis and/or Prognosis Uses and Dedicated Kits
Risk to relapse and/or to develop a tumor metastasis

Another object of the invention is an in vitro method for diagnosing or predicting the risk to relapse and/or to develop a tumor metastasis in a subject treated by its conventional treatment comprising an anti-angiogenic compound, characterized in that said method comprises a step of contacting a biological sample of said subject with an anti-VEGFC antibody of the invention, it being possible for said antibody to be, if necessary, labelled.

In a particular embodiment, the subject is affected by a cancer or a disease related to undesirable lymphatic cell proliferation and/or migration and is treated by a conventional treatment. Such conventional treatments are disclosed above. In a more particular embodiment, the conventional treatment comprises an anti-angiogenic treatment, in particular an anti-VEGF antibody distinct from an anti-VEGFC antibody according to the invention.

In particular, the method comprises a step of determining the expression and/or the level of expression of at least one human proteolytically cleaved form of VEGFC in a biological sample, by detecting and/or quantifying the binding of said antibody according to the invention with the mature form of VEGFC that specifically binds to VEGF-R3 expressed on lymphatic endothelial cells.

So the invention also relates to an in vitro method for diagnosing or predicting the risk to relapse and/or to develop a tumor metastasis in a subject treated by its conventional treatment comprising an anti-angiogenic compound, said method comprising:
i) determining the expression and/or the level of expression of at least one human proteolytically cleaved form of VEGFC in a biological sample of said subject using at least one anti-VEGFC antibody and/or fragment thereof according to the invention;
ii) comparing said level of expression determined at step i) to a control level and determining if said subject has a risk to relapse and/or to develop a tumor metastasis.

For example, step i) can be performed by an ELISA assay, wherein the anti-VEGFC antibody or fragment thereof of the invention is immobilized on a microtiter plate, said plate being thereafter incubated with at least one labelled secondary antibody directed proteolytically cleaved form of VEGFC, which recognize the antibody and/or fragment thereof according to the invention, in appropriate conditions well-known in the art.

In a preferred embodiment, the method of the invention comprises the step ii) of comparing said level of expression determined at step i) to a control level and determining if said level of expression determined at step i) is significantly higher than said control level; said significantly higher level of expression indicates that the subject is has a risk to relapse and/or to develop a tumor metastasis; wherein said control level is the expression level of said proteolytically cleaved form of VEGFC determined in at least a biological sample from an healthy subject, or from a subject who is not affected with a disease related to undesirable lymphatic cell proliferation and/or migration or a cancer.

The term “biological sample” encompasses a variety of sample types obtained from an organism that may be used in a diagnostic or prognostic assay. The term encompasses blood and other liquid samples of biological origin, solid tissue samples, such as a biopsy specimen, or tissue cultures or cells derived there from and the progeny thereof.

Additionally, the term may encompass circulating tumors or other cells. The term specifically encompasses a clinical sample, and further includes cells in cell culture, cell supernatants, cell lysates, serum, plasma, urine, amniotic fluid, biological fluids including aqueous humour and vitreous for eyes samples, and tissue samples. The term also encompasses samples that have been manipulated in any way after procurement, such as treatment with reagents, solubilisation, or enrichment for certain components.

Advantageously, the biological sample can be selected from the group comprising a bodily fluid, a fraction thereof, tissue extract and, cell extract. Particularly the biological sample can be selected from the group comprising plasma sample and tumor extract. The method for detecting in a sample the proteolytically cleaved form of VEGFC that specifically binds to VEGF-R3 according to the invention can be based on various techniques, well known by one skilled in the art, including, but not limited to:
a western blot assay (the proteolytically cleaved form of VEGFC present in a cell lysate or in a solution being immobilized on a membrane, the said membrane being thereafter incubated with the antibody of the invention, preferably labelled, in appropriate conditions well-known in the art),
an ELISA assay (the proteolytically cleaved form of VEGFC being immobilized on a microtiter plate, the said plate being thereafter incubated with the antibody of the invention, preferably labelled, in appropriate conditions well-known in the art),
an immunohistochemistry assay (the recombinant antibody, preferably labelled, being used to stain a sample containing fixed cells or tissues expressing the VEGFC),
a flow cytometry assay (the recombinant antibody, preferably labelled, being used to stain a sample containing fixed or living cells expressing the VEGFC, in appropriate conditions well-known in the art).

In one embodiment of the method for detecting in a sample a proteolytically cleaved form of VEGFC according to the invention, the antibody of the invention is coated on a solid support.

These detection techniques are well-described in Sambrook, Fritsch and Maniatis—“Molecular Cloning—A Laboratory Manual” Second Edition Cold Spring Harbor Laboratory, 1989. Any other detection techniques requiring the use of an antibody are herein encompassed. The presence and eventually the amount of said cells expressing proteolytically cleaved form of VEGFC in said sample can be determined thanks to these techniques. Some of these techniques require labelling the antibody of the invention with a detectable marker, preferably a fluorescent or a luminescent marker, as disclosed above.

Such in vitro method permits to identify and/or select subjects (patients) that may benefit of a combined therapy comprising anti-VEGFC antibody of the invention and anti-angiogenic compound.
Diagnostic and/or prognostic method of a disease related to undesirable lymphatic cell proliferation and/or migration

The invention also relates to in vitro or ex vitro diagnostic and/or prognostic method of a disease related to undesirable lymphatic cell proliferation and/or migration, in particular of clear cell renal cell carcinoma, in a subject, said method comprising:
i) determining the expression and/or the level of expression of at least one human proteolytically cleaved form of VEGFC in a biological sample of said subject using at least one anti-VEGFC antibody and/or fragment thereof according to the invention;
ii) comparing said level of expression determined at step i) to a control level and determining if said subject is affected with a disease related to undesirable lymphatic cell proliferation and/or migration, more particularly with a clear cell renal cell carcinoma.

In particular, step i) can be performed by further using at least one labelled secondary antibody directed to proteolytically cleaved form of VEGFC that recognizes the anti-VEGFC antibody and/or fragment thereof of the invention.

For example, step i) can be performed by an ELISA assay, wherein the anti-VEGFC antibody or fragment thereof of the invention is immobilized on a microtiter plate, said plate being thereafter incubated with at least one labelled secondary antibody directed proteolytically cleaved form of VEGFC, which recognize the antibody and/or fragment thereof according to the invention, in appropriate conditions well-known in the art.

In a preferred embodiment, the method of the invention comprises the step ii) of comparing said level of expression determined at step i) to a control level and determining if said level of expression determined at step i) is significantly higher than said control level; said significantly higher level of expression indicates that the subject is affected with a disease related to undesirable lymphatic cell proliferation and/or migration, more particularly with clear cell renal cell carcinoma; wherein said control level is the expression level of said proteolytically cleaved form of VEGFC determined in at least a biological sample from an healthy subject, or from a subject who is not affected with a disease related to undesirable lymphatic cell proliferation and/or migration, particularly with clear cell renal cell carcinoma.
In vitro method to determine a bad outcome of a disease related to undesirable lymphatic cell proliferation and/or migration

The invention also relates to in vitro or ex vitro method to determine a bad outcome of a disease related to undesirable lymphatic cell proliferation and/or migration, more particularly of clear cell renal cell carcinoma, in a subject, said method comprising:
a) determining the expression and/or the level of expression of at least one human proteolytically cleaved form of VEGFC in a biological sample of said subject using at least one antibody and/or fragment thereof according to the invention.

The method of the invention can further comprise the step b) of comparing said level of expression determined at step a) to a control level and determining if said subject is or not undergoing a bad outcome of a disease related to undesirable lymphatic cell proliferation and/or migration, more particularly of a clear cell renal cell carcinoma.

Suitable “control level” include the expression level of said proteolytically cleaved form of VEGFC determined in at least one reference sample and a reference threshold value.

A “reference sample” is a biological sample from a subject with a known state of disease related to undesirable lymphatic cell proliferation and/or migration, more particularly a known clear cell clear cell renal cell carcinoma state, or from a healthy subject, or from a subject who is not affected with a disease related to undesirable lymphatic cell proliferation and/or migration, more particularly with clear cell renal cell carcinoma.

Preferably, the control level is the mean expression levels of said proteolytically cleaved form of VEGFC determined in several reference samples, from several subjects.

Preferably, the reference sample is the same type of biological sample (i.e. a biological sample of corresponding physiological nature) than said biological sample of step i) or a). In a preferred embodiment, the method of the invention comprises the step b) of comparing said level of expression determined at step a) to a control level and determining if said level of expression determined at step a) is significantly higher than said control level; said significantly higher level of expression indicates a bad outcome of disease related to undesirable lymphatic cell proliferation and/or migration, more particularly of clear cell renal cell carcinoma in said subject; wherein said control level is the expression level of said proteolytically cleaved form of VEGFC determined in at least a biological sample from a healthy subject, or from an subject who is not affected with a disease related to undesirable lymphatic cell proliferation and/or migration, particularly with a pathological angiogenesis disease, more particularly with clear cell renal cell carcinoma.

Suitable “control level” include the expression level of said proteolytically cleaved form of VEGFC determined in the same type of biological sample of a healthy subject, of a subject who is not affected with a disease related to undesirable lymphatic cell proliferation and/or migration, more particularly with clear cell renal cell carcinoma.

Preferably, the control level is the mean expression levels of said proteolytically cleaved form of VEGFC determined in the same type of biological sample of several healthy subjects, of several subjects who are not affected with a disease related to undesirable lymphatic cell proliferation and/or migration, more particularly with clear cell renal cell carcinoma.

Said significant higher level of expression of said proteolytically cleaved form of VEGFC can corresponds to an increase of expression of at least more than 10%, more than 15%, more than 20%, more than 25%, more than 30%, preferably more than 35% of said control level.

Such a reference threshold value may vary depending on the type of tested biological sample and the method used for determining the level of expression of said proteolytically cleaved form of VEGFC. However, for particular experimental conditions (same type of tested biological sample, same method for determining the level of expression of said at least one chemokine), said threshold value may be determined based on a reference pool of patients comprising both a population of patients with stable disease related to undesirable lymphatic cell proliferation and/or migration, more particularly stable clear cell renal cell carcinoma (alive patients) and a population of patients undergoing a bad outcome of disease related to undesirable lymphatic cell proliferation and/or migration, in particular clear cell renal cell carcinoma (deceased patients). By measuring the levels of expression of said proteolytically cleaved form of VEGFC of these patients, a reference threshold value T can be determined by the following features:
all or most reference patients with stable disease related to undesirable lymphatic cell proliferation and/or migration, more particularly stable clear cell renal cell carcinoma (alive patients) have at least proteolytically cleaved form of VEGFC expression level values inferior to T; and
all or most reference patients undergoing a bad outcome of disease related to undesirable lymphatic cell proliferation and/or migration, more particularly of clear cell renal cell carcinoma (deceased patients) have at least proteolytically cleaved form of VEGFC expression level values superior to T.

In this case, if said level of expression determined at step i) is significantly higher than said threshold value T, it indicates a bad outcome of disease related undesirable lymphatic cell proliferation and/or migration, more particularly of clear cell renal cell carcinoma in said subject.

The term “determination” as used herein may mean both qualitatively detecting and quantifying.

The term “expression” generally refers to the process by which a polynucleotide sequence undergoes successful transcription and translation such that detectable levels of the amino acid sequence or protein are expressed. In certain contexts, herein, expression refers to the production of mRNA. In other contexts, expression refers to the production of protein or fragments thereof. The fragments may be produced via enzymatic cleavage or biological processes characteristic of normal or diseased conditions.

Any of a variety of known methods may be used for detection of said proteolytically cleaved form of VEGFC in said biological sample of said individual, including, but not limited to, immunoassay, using antibody or fragment thereof according to the invention, e.g., by enzyme-linked immunosorbent assay (ELISA), radioimmunoassay (RIA), and the like.
Kit for carrying diagnostic and/or prognostic methods

The invention also relates to a kit for carrying out the in vitro method for diagnosing or predicting the risk to relapse and/or to develop a tumor metastasis in a subject treated by its conventional treatment comprising an anti-angiogenic compound, said kit comprises at least one anti-VEGFC antibody and/or fragment thereof according to the invention. In a particular embodiment, the invention also relates to a kit for carrying out the in vitro diagnostic and/or prognostic method of a disease related to undesirable lymphatic cell proliferation and/or migration, more particularly of clear cell renal cell carcinoma, in a subject, said kit comprises at least one anti-VEGFC antibody and/or fragment thereof according to the invention.

The said kits may provide additional components that are useful in methods of the invention, including, but not limited to, buffers, developing reagents, labels, reacting surfaces, means for detection, control samples, standards, instructions, and interpretive information for determining if a subject is affected with a disease related to undesirable lymphatic cell proliferation and/or migration, more particularly of clear cell renal cell carcinoma. The kit can also comprise:
at least one reagent for detecting said anti-VEGFC antibody or fragment thereof according to the invention.
Personalized treatment

The invention also relates to a method comprising:
I′) diagnosing or predicting the risk to relapse and/or to develop a tumor metastasis in a subject treated by its conventional treatment comprising an anti-angiogenic compound, in particular according to the method of the invention disclosed above; and
ii″) treating subject having the risk to relapse and/or to develop a tumor metastasis in a subject treated by its conventional treatment comprising an anti-angiogenic compound, with a pharmaceutical composition or combination of the invention as disclosed above.

In particular, said method comprises:
I′) diagnosing or predicting the risk to relapse and/or to develop a tumor metastasis in a subject treated by its said conventional treatment, comprising the determination of the expression and/or the level of expression of at least one human proteolytically cleaved form of VEGFC in a biological sample of said subject, and
ii″) treating a subject having the risk to relapse and/or to develop a tumor metastasis in a subject treated by its conventional treatment comprising an anti-angiogenic compound, with a pharmaceutical composition or combination of the invention, comprising the administration of at least an anti-VEGFC antibody or fragment thereof according to the invention; or a combination of said anti-VEGFC antibody with at least an anti-angiogenic compound and optionally another compound selected from anti-tumor compound and anti-inflammatory compound, for simultaneous, separate or sequential administration.

The invention also relates to a method comprising:
I″) diagnosing a disease related to undesirable lymphatic cell proliferation and/or migration, more particularly of clear cell renal cell carcinoma, in a subject, in particular according to the in vitro diagnostic method of the invention; and
ii″) treating subject affected with a disease related to undesirable lymphatic cell proliferation and/or migration, more particularly of clear cell renal cell carcinoma, in a subject, with a pharmaceutical composition or combination of the invention as disclosed above.

In particular, said method comprises:
i″) diagnosing a disease related to undesirable lymphatic cell proliferation and/or migration, more particularly of clear cell renal cell carcinoma, in a subject, comprising the determination of the expression and/or the level of expression of at least one human proteolytically cleaved form of VEGFC in a biological sample of said subject; and
ii″) treating a subject affected with a disease related to undesirable lymphatic cell proliferation and/or migration, more particularly of clear cell renal cell carcinoma, with a pharmaceutical composition or combination of the invention, comprising the administration of at least an anti-VEGFC antibody or fragment thereof according to the invention; or a combination of said anti-VEGFC antibody with at least an anti-angiogenic compound and optionally another compound selected from anti-tumor compound and anti-inflammatory compound, for simultaneous, separate or sequential administration.

The invention also relates to a method comprising:
I′″) determining a bad outcome of a disease related to undesirable lymphatic cell proliferation and/or migration, more particularly of clear cell renal cell carcinoma in a subject, in particular according to the method of the invention;

Ii′″) treating a subject who is undergoing a bad outcome of a disease related to disease related to undesirable lymphatic cell proliferation and/or migration, more particularly of clear cell renal cell carcinoma, with a pharmaceutical composition or combination of the invention, comprising the administration of at least an anti-VEGFC antibody or fragment thereof according to the invention; or a combination of said anti-VEGFC antibody with at least an anti-angiogenic compound and optionally another compound selected from anti-tumor compound and anti-inflammatory compound, for simultaneous, separate or sequential administration.

In particular, said method comprises:
I′″) determining a bad outcome of a disease related to undesirable lymphatic cell proliferation and/or migration, more particularly of clear cell renal cell carcinoma in a subject, comprising the determination of the expression and/or the level of expression of at least one human proteolytically cleaved form of VEGFC in a biological sample of said subject; and
Ii′″) treating a subject who is undergoing a bad outcome of a disease related to disease related to undesirable lymphatic cell proliferation and/or migration, more particularly of clear cell renal cell carcinoma, with a pharmaceutical composition or combination of the invention, comprising the administration of at least an antibody or fragment thereof according to the invention; or a combination of said antibody with at least an anti-angiogenic compound and optionally another compound selected from anti-tumor compound and anti-inflammatory compound, for simultaneous, separate or sequential administration.

### BRIEF DESCRIPTION OF THE FIGURES

FIG. 1: The VEGFC sequence

FIG. 2: Flow chart of the selection of anti-VEGFC hybridomas.

FIG. 3: Isotyping of the different antibodies

FIG. 4: Effect of antibodies 1E9 on the phosphorylation of VEGFR3 and VEGFR2 in endothelial cells: ELISA assay for VEGFR3 (a) or immunobloting for VEGFR2 activation (b).

FIG. 5: Effect of antibodies 1E9 on the viability of vascular endothelia cells HuVEC (FIG. 5a) and on the migration of lymphatic endothelial cells (LEC) (FIG. 5b).

FIG. 6: Effect of antibodies 1E9 on the proliferation of kidney and breast tumor cells expressing VEGFC and its receptor/co-receptor through NRP2 signaling: effect on proliferation of kidney and breast tumor cells—FIG. 6a, and proliferation curves of 786-O and 786-O_KO_NRP2-FIG. 6b.

FIG. 7: The nucleotide and the protein sequences of the variable regions of the light and heavy chains of the chimeric antibodies.

FIG. 8: Effect of antibodies 1E9 alone or in combination with Bevacizumab (BVZ) on experimental RCC: effect on the growth (FIG. 8a) and on the tumor weight (FIG. 8b).

FIG. 9: Effect of bevacizumab on the expression of VEGFC on 786-O and A498 cells.

The present invention will be explained with examples in the following text, but the technical scope of the present invention is not limited to these examples.

### EXAMPLES

### Example 1: Preparation of Anti-VEGFC Monoclonal Antibodies

CHO expressing the VH and VL chains (cloning of the corresponding gene in pFUSE vectors (InVivoGen) are cultivated in F12 medium with 5% low IgG serum. Cells were cultivated until they reached confluency. Then medium was replaced by a production medium containing 1.25% low IgG serum. The conditioned medium is recovered 20 days after for antibody purification. Cell supernatant is loaded on a HiTrap™ Protein G HP-column (flow 0.2 to 1 ml/minute for a 5 ml column). Fixed antibodies were desorbed in the elution buffer (1 M Tris-HCl pH 2.7) and immediately neutralized in neutralizing buffer (1 M Tris pH 9), 100 μl per ml of fraction).

Cloning of VEGFC in pGEX-6P-3

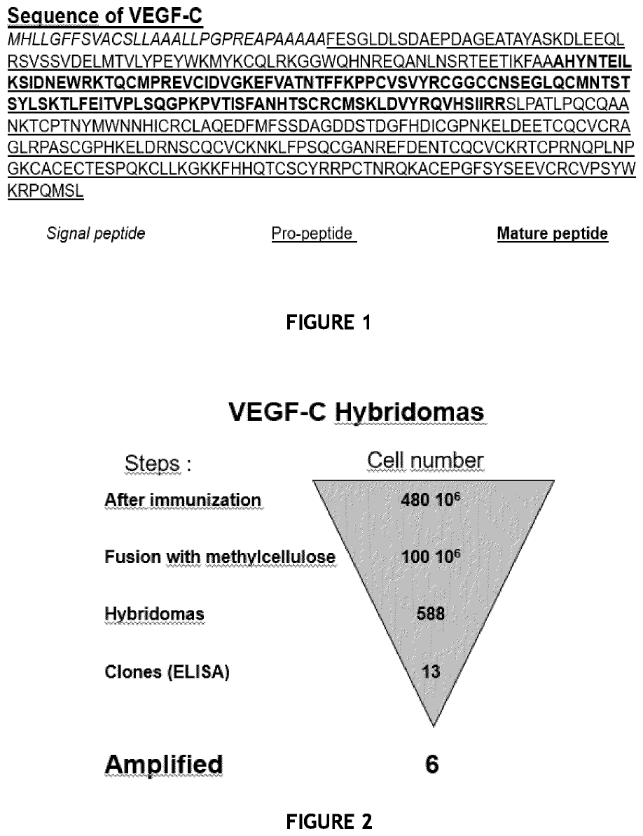

The immunogen comprising the sequence coding for the mature peptide (SEQ ID NO:1) of the VEGFC that specifically stimulates VEGFR3, was subcloned in the pGEX6P3 vector. The FIG. 1 presents the sequence of VEGF-C with the mature peptide in bold characters.

Production and Purification of a Fusion Protein GST-VEGFC

This plasmid was transformed in BL21 bacteria for the production of a GST-VEGFC fusion protein.

Mice Immunization, Cell Fusion and Hybridoma Screening

Mice were injected several times with the immunogen comprising the sequence coding for the mature peptide (SEQ ID NO:1) of the VEGFC.

The screening of specific hybridoma producing anti-VEGFC antibodies was based on the ability of the hybridoma to recognize the VEGFC antigen used in the immunization protocol by ELISA. The flow-chart of the experiment is described in FIG. 2.

Serum from each mouse was tested by ELISA against the VEGFC antigen harvested from the mice with the strongest immune response.

Supernatants of hybridomas were screened by ELISA for the ability to bind the VEGFC antigen. Several rounds of screening were performed to ensure that only hybridomas stably producing antibodies recognizing the VEGFC antigen were selected.

Thirteen clones were selected for their ability to produce specific antibodies and the six best were amplified.

Isotyping of the Different Antibodies

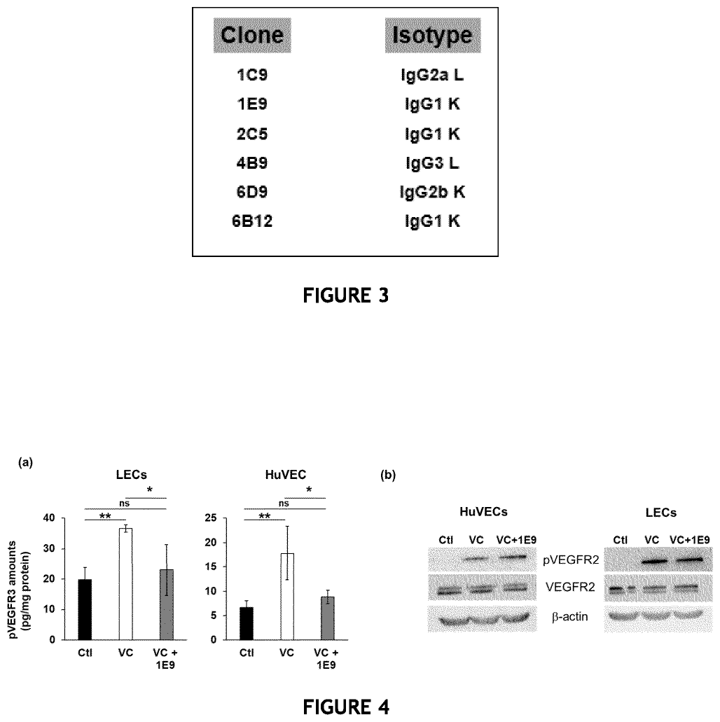

Isotyping of the six clones was performed using specific commercially available kits, and the results were presented in FIG. 3.

Efficacy of the Different Antibodies

Cell Lines and Culture Conditions

HuVECs (Human umbilical vein endothelial cells), LECs (Lymphatic endothelial cells), 786-O, A498 and MDA-MB231 were purchased from the American Tissue Culture Collection (ATCC). 786-O_#NRP2 knock-out (KO) cells were generated in the laboratory as previously described in Dumond, A (2021). Cells were cultured as indicated by ATCC and as already described in Signoretti, S et al. (2018) and Grepin, Ret al. (2014).

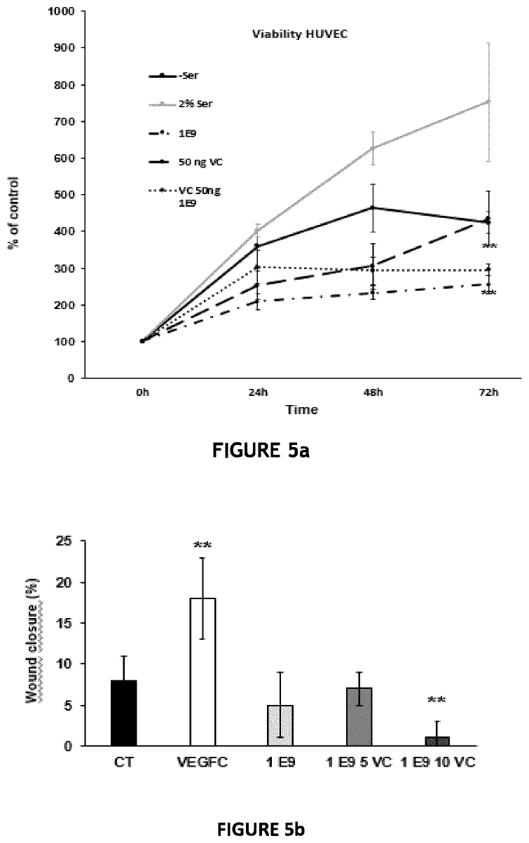

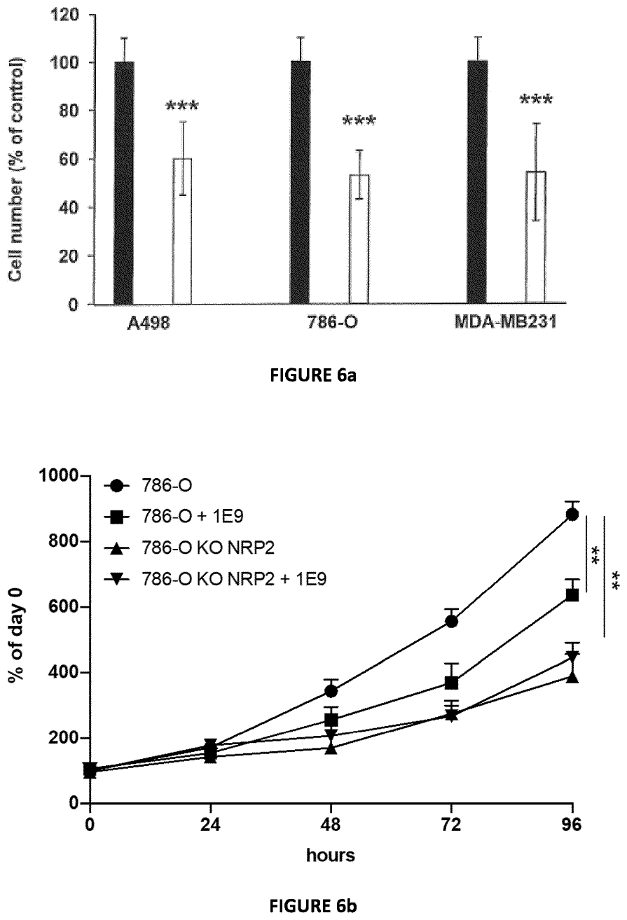

The different antibodies (six clones) were tested for their ability to inhibit the phosphorylation of VEGFR3 but not of VEGFR2 in endothelial cells (FIG. 4), the viability of endothelial cells (HuVEC) expressing VEGFR2 (FIG. 5a), the migration of lymphatic endothelial cells (LECs) expressing VEGFR3 (FIG. 5b) and the proliferation of tumor cells (FIG. 6) considering that aggressive cancer cells, including RCC cells, express VEGFC and their receptors (VEGFR2/3) or their co-receptors the neuropilins especially neuropilin 2. The effects of the antibodies 1E9 that were the most potent were described in the said figures. Of note VEGFR2 and VEGFR3 can be stimulated by VEGFC.

Effect of Antibodies 1E9 on the Phosphorylation of VEGFR3 and of VEGFR2 in Endothelial Cells

For 786-O and 786-O_#NRP2-KO, cells were incubated with an irrelevant or 1E9 antibodies (10 μg/mL) for 96 h. Cell proliferation was monitored during 96 h by cell counting. Results are expressed as % of day 0.

An ELISA assay for VEGFR3 (FIG. 4a) or immunoblotting for VEGFR2 activation (FIG. 4b) were carried out. Statistical significance and P values were determined with the two tailed t test (*p<0.05; ** p<0.01; *** p<0.001).

Immunoblotting

HuVECs and LECs cells were starved for 2 h and then treated for 20 min with VEGFC (100 ng/mL) in presence or not of 1E9 antibodies (10 μg/mL). Cells were lysed in Laemmli buffer containing 2% SDS, 10% Glycerol, 60mM Tris-HCl, 1X Halt' phosphatase inhibitor cocktail (Thermo Fischer). DNA was fragmented by sonification. Lysates supplemented with 0.002% bromophenol blue and 100 mM DTT were heated to 96° C., separated by SDS-PAGE, and transferred to PVDF membranes (Millipore). Membranes were probed with the following antibobies (pVEGFR2 (Tyr1175), CST, 2478S; VEGFR2, CST, 2479S and β-actin (D6A8) CST, 8457.

ELISA Assay

The production of VEGFC following bevacizumab exposure was determined in vitro in 786-O and A498 cells by ELISA using R&D systems ELISA kit. Cells were plated in 6-well plates and treated with bevacizumab (10 μg/mL) for 48 h. Supernatants were collected and ELISA performed according to the manufacturer recommendations. Results are expressed as pg/mL/millions of cells. The activation of VEGF receptor 3 (VEGFR3) was performed by ELISA using Human phospho-VEGFR3 DuoSet IC ELISA, R&D systems). HuVECs and LECs cells were starved for 2 h and then treated for 20 min with VEGFC (100 ng/mL) in presence or not of 1E9 antibodies (10 μg/mL). Results are expressed as pg of phospho-VEGFR3/μg of proteins

The results presented in FIG. 4 showed that 1E9 antibodies decreased the VEGFC-dependent phosphorylation of VEGFR3 but not VEGFR2.

Effect of Antibodies 1E9 on the Viability of Vascular Endothelia Cells.

Human vascular endothelial cells (HUVEC) viability was assessed as already described using the ADAM technology [22]. Cells were incubated during the indicated times in a medium specific for endothelial cells (Promocell) without growth factor (-ser), in the presence of 2% fetal bovine serum (−2%), in the presence of 50 ng/ml of the non-maturated form of VEGFC that can stimulate VEGFR2 and VEGFR3 (VC 50), in the presence of 10 mg/ml of 1E9 antibodies (1E9) and 50 ng/ml of VEGFC plus 10 mg/ml of 1E9 antibodies (Vc 50 n 1E9). **p<0.01.

Briefly, human endothelial cells (HuVEC) kept in low concentration of serum (0.1%) were stimulated or not with serum (2%) or VEGFC (50 ng/ml) in the presence or not of 1E9 antibodies. 1E9 antibodies decreased the basal and VEGFC-stimulated viability of HuVEC cells (p<0.01).

The results presented in FIG. 5a showed that antibodies 1E9 inhibit the viability of vascular endothelial cells expressing VEGFC.

Effect of Antibodies 1E9 on the Migration of Lymphatic Endothelial Cells (LEC).

A confluent LEC monolayer was scratched with a yellow tip. The wound closure was assessed after 10 hours in absence of growth factors (CT), in the presence of 50 ng/ml of VEGFC (VEGFC), in control condition plus 5 μg/ml of 1E9 (1 E9), and in the presence of 5 or 10 μg/ml 1E9 plus 50 ng/ml of VEGFC (1E9 5 or 10 VC). ** p<0.01.

The results presented in FIG. 5b showed that 1E9 antibodies inhibit the migration of lymphatic endothelial cells. Therefore, 1E9 antibodies inhibited the VEGFC-dependent migration of lymphatic endothelial cells.

Effect of Antibodies 1E9 on the Proliferation of Kidney and Breast Tumor Cells Expressing VEGFC and its Receptor/Co-Receptor.

Several papers showed that tumor cells aberrantly overexpress VEGF and VEGFC and their receptors (VEGFR1, VEGFR2) creating autocrine proliferation loops. However, ccRCC cells do not exert these autocrine loops via VEGFR2 or VEGFR3 but depend on their respective co-receptors Neuropilin 1 and Neuropilin 2. Triple negative breast cancer cells overexpress VEGF and VEGFC but do not express VEGFR. Their proliferation greatly depends on a VEGFNEGFC/NRP1/NRP2 autocrine proliferation loops. Considering the potent effects of the 1 E9 antibodies on the VEGFC-dependent signaling, we hypothesized that they should inhibit the proliferation of tumor cells presenting such autocrine loops. Therefore, we tested the 1E9 antibodies on ccRCC (A498 and 786-O, Neuropilin 1 and 2 positive) and breast (MDA-MB231, VEGFR2 and Neuropilin 1 positive) tumor cells.

Kidney (A498, 786-O) and breast (MDA-MB231) cancer cells proliferation was assessed by MTT assays after 48 h of incubation with 10 μg/ml of purified 1E9 antibodies in DMEM medium containing 2% fetal bovine serum. The three independent cell lines express high amounts of VEGFC (around 1 ng/ml/10⁶ cells). Kidney cells only express the VEGFC co-receptor neuropilin 2 and the MDA-MB231 only express the VEGFC receptor VEGFR3. *** p<0.001.

For the proliferation curves of 786-O and 786-O_KO_NRP2 (FIG. 6b), cells were incubated with 1E9 antibodies and counted at the indicated times. ** p<0.01, *** p<0.001. The results presented in FIG. 6 showed that antibodies 1E9 inhibit the proliferation of kidney and breast tumor cells expressing VEGFC and its receptor/co-receptor. At 10 μg/mL, 1E9 antibodies decreased by 40% the proliferation of cancer cells (FIG. 6a). These effects on 786-O cell proliferation were found to be dependent on NRP2 signaling (FIG. 6b).

Therefore, in addition to its impact on angiogenesis/lymphangiogenesis (inhibition of survival/proliferation/migration of vascular and lymphatic endothelial cells), 1E9 antibodies have a strong impact on tumor cell proliferation.

### Example 2: Preparation of Chimeric Antibodies

Two monoclonal antibodies produced by 1E9 hybridoma were cloned and sequenced using techniques known to those skilled in the art. The variable regions of these 1E9 antibodies were sequenced (heavy and light chains). The CDR of these two antibodies were fused to human IgG1 light and heavy chains to obtain chimeric antibodies that can be used in the clinic. Expression vector were transfected in CHO cells to obtain stable clones expressing the chimeric antibodies. The chimeric antibodies recognized with a high affinity the human VEGFC.

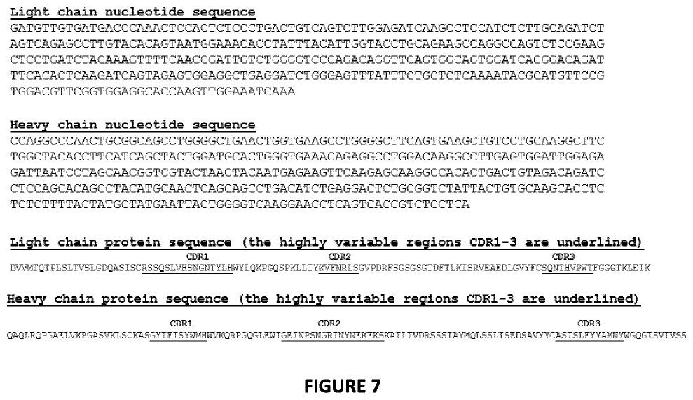

In particular, the variable region of the light and heavy chain of these antibodies were sequenced and cloned in the pFUSE2ss-CLlg-hk and pFUSEss-CHIg-hG1 vectors (In vivoGen San Diego, Calif. 92121—USA) respectively. The murine parts of the monoclonal antibodies were fused to the constant light and heavy chain of human IgG1 antibodies. The vectors were transfected in CHO cells sequentially. CHO clones thank can grow in suspension and that produced the VEGFC antibodies were obtained. The yield of production is about 10 mg of chimeric antibodies per liter of medium without serum. Equivalent results for anti-proliferation and anti-migration abilities were obtained with the murine monoclonal antibodies and the chimeric antibodies. The nucleotide and the protein sequences of the variable regions of the light and heavy chains are the following (FIG. 7).

SEQ ID NO: 2: nucleic sequence of the variable region heavy chain

SEQ ID NO: 3: nucleic sequence of the variable region light chain

SEQ ID NO: 4: amino acid sequence of the variable region heavy chain

SEQ ID NO: 5: amino acid sequence of the variable region light chain

SEQ ID NO: 6: heavy chain CDR1 (CDR-H1)

SEQ ID NO: 7: heavy chain CDR2 (CDR-H2)

SEQ ID NO: 8: heavy chain CDR3 (CDR-H3)

SEQ ID NO: 9: light chain CDR1 (CDR-L1)

SEQ ID NO: 10: light chain CDR2 (CDR-L2)

SEQ ID NO: 11: light chain CDR3 (CDR-L3)

### Example 3: Efficacy of Anti-VEGFC Antibodies on Experimental Models of ccRCC

Effect of Antibodies 1E9 on the Growth of Experimental RCC.

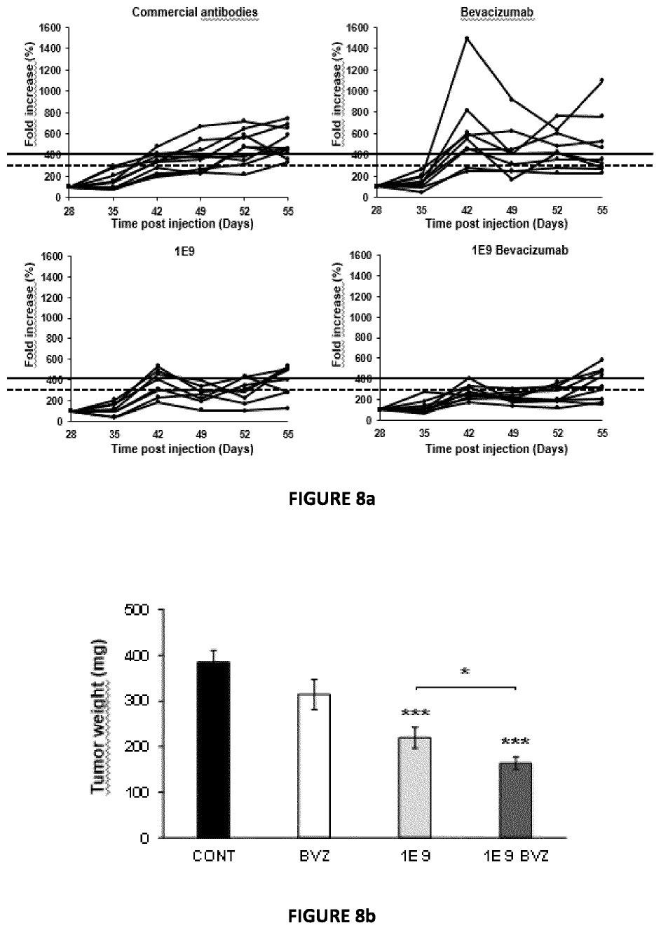

Chimeric antibodies were used to performed in vivo experiments. For these experiments, the efficacy of anti-VEGFC antibodies alone or combined with the anti-VEGF antibodies used in the clinic (Bevacizumab/Avastin) was tested on the growth of experimental RCC in nude mice (FIG. 8).

3×10⁶ 786-O RCC cells were subcutaneously injected in the flank of nude mice. Tumor growth was evaluated with a caliper as already described [5]. 28 days after tumor cell injection, the average tumor size in each group was determined and used as the reference tumor size before treatments (100%). Treatments started at 28 days for mice bearing tumors ranging between 70 and 100 mm³ in size (5 mg/kg twice a week during the indicated times). Tumor size in mm³ was then evaluated for the indicated time points and reported to the reference size at 28 days for each tumor (10 per group). The fold increase for each tumor was reported in the figure. High and low thresholds were indicated by black (plain dotted) lines respectively. Statistics are indicated; *, p<0.05; ***, p<0.001.

Effect of Antibodies 1E9 Alone or in Combination with Bevacizumab (BVZ) on Tumor Weight

Tumor weight at the end of the experiments was significantly lower for tumors of mice treated with the 1E9 antibodies alone or combined with anti-VEGF antibodies (FIG. 8b). So, as we previously described, bevacizumab (BVZ) had a trend to accelerate tumor growth (FIG. 8a) although it did not modify tumor weight at sacrifice (FIG. 8b) as compared to control tumors from mice receiving irrelevant antibodies. 1E9 antibodies (1E9) decreased the growth of 786-O xenografts (FIG. 8a) and their weight (FIG. 8b). The combination of 1E9 antibodies with bevacizumab (1E9+BVZ) inhibited more efficiently tumor growth (FIG. 8a). Tumor weight was significantly decreased as compared to the control condition and as compared to 1E9 antibodies alone (FIG. 8b). ***, p<0.001. These results suggest that 1E9 antibodies had a therapeutic efficacy at least on experimental models but that they also revert the detrimental effects of bevacizumab alone that we previously described.

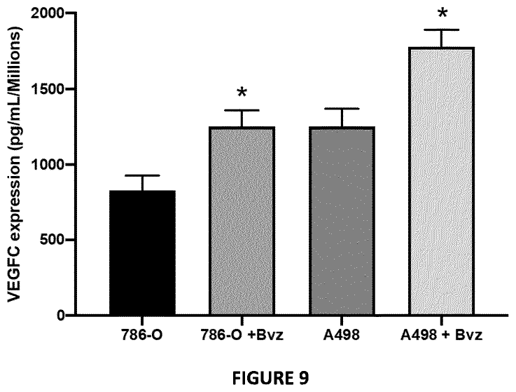

Furthermore, FIG. 9 shows that the treatment of 786-O and A498 cells in vitro with bevacizumab for 48 h significantly increased the expression of VEGFC. *, p<0.05.

The additive effect of anti-VEGFC plus anti-VEGF antibodies was consistent with previous results showing that resistance to anti-VEGF antibodies or to tyrosine kinase inhibitors of the VEGFNEGFR signaling was dependent on VEGFC and the subsequent development of lymphatic vessels [5, 6].

So, the anti-VEGFC antibodies of the invention inhibit the growth of experimental kidney tumors in nude mice. Whereas anti-VEGF antibodies have no effect or even favor experimental tumor growth in this model, the anti-VEGF plus anti-VEGFC combination have an additive effect on the inhibition of tumor growth.

Therefore, these antibodies may constitute a new therapeutic strategy for metastatic kidney tumors.

Although tyrosine kinase inhibitors TKI or anti-VEGF reduced transiently the size of distant metastasis, they promote at the same time the development of an alternative vessel network participating in further metastatic dissemination. It was also observed that one common denominator of the response of tumor cells to several types of treatments was the induction of VEGFC; i) platinum salts for lung cancer cells, oral squamous cell carcinoma, melanoma, MOB ii) taxanes for breast or prostate cancers; iii) radiotherapy for oral squamous cell carcinoma (OSCC) [23]. Hence, a bundle of evidence links tumor resistance to a VEGFC-dependent lymphangiogenesis and further metastatic dissemination. Although the experiments illustrated above are centered on RCC, anti-VEGFC antibodies of the invention combined with the reference treatment of a specific tumor, may be proposed in the first line or at relapse.

In addition, bevacizumab was also approved for the treatment of breast cancers but it lost its FDA approval for insufficient benefits. We observed that several cell lines representative of triple negative breast cancers including MDA-MB231 overexpress VEGF and VEGFC. Hence, combining anti-VEGF and anti-VEGFC appears relevant for the treatment of could triple negative breast cancer for which the VEGFCNEGFR3/NRP2 signaling is detrimental.

### REFERENCES

1. Giuliano S, Pages G. Mechanisms of resistance to anti-angiogenesis therapies. Biochimie 2013; 95:1110-9.
2. Alitalo K, Tammela T, Petrova T V. Lymphangiogenesis in development and human disease. Nature 2005; 438:946-53.
3. Alitalo A, Detmar M. Interaction of tumor cells and lymphatic vessels in cancer progression. Oncogene 2012; 31:4499-508.
4. Wang C A, Jedlicka P, Patrick A N, et al. SIX1 induces lymphangiogenesis and metastasis via upregulation of VEGFC in mouse models of breast cancer. J Clin Invest 2012; 122: 1895-906.
5. Dufies M, Giuliano S, Ambrosetti D, et al. Sunitinib Stimulates Expression of VEGFC by Tumor Cells and Promotes Lymphangiogenesis in Clear Cell Renal Cell Carcinomas. Cancer Res 2017; 77:1212-26.
6. Grepin R, Guyot M, Jacquin M, et al. Acceleration of clear cell renal cell carcinoma growth in mice following bevacizumab/Avastin treatment: the role of CXCL cytokines. Oncogene 2012; 31:1683-94.
7. Escudier B, Bellmunt J, Negrier S, et al. Phase III trial of bevacizumab plus interferon alfa-2a in patients with metastatic renal cell carcinoma (AVOREN): final analysis of overall survival. J Clin Oncol 2010; 28:2144-50.
8. Joukov V, Pajusola K, Kaipainen A, et al. A novel vascular endothelial growth factor, VEGFC, is a ligand for the Flt4 (VEGFR-3) and KDR (VEGFR-2) receptor tyrosine kinases. EMBO J 1996; 15:290-98.
9. Chothia C, Lesk A M, Tramontano A, et al. Conformations of immunoglobulin hypervariable regions. Nature 1989; 342:877-83.
10. Chothia C, Lesk A M. Canonical structures for the hypervariable regions of immunoglobulins. J Mol Biol 1987; 196:901-17.
11. Kozbor D, Roder J C. The production of monoclonal antibodies from human lymphocytes. Immunol Today 1983; 4:72-9.
12. Roder J C, Cole S P, Kozbor D. The EBV-hybridoma technique. Methods Enzymol 1986; 121:140-67.
13. Huse W D, Sastry L, Iverson S A, et al. Generation of a large combinatorial library of the immunoglobulin repertoire in phage lambda. Science 1989; 246:1275-81.
14. Duroux P, Kaas Q, Brochet X, et al. IMGT-Kaleidoscope, the formal IMGT-ONTOLOGY paradigm. Biochimie 2008; 90:570-83.
15. Giudicelli V, Lefranc M P. Ontology for immunogenetics: the IMGT-ONTOLOGY. Bioinformatics 1999; 15:1047-54.
16. Ehrenmann F, Kaas Q, Lefranc MP. IMGT/3Dstructure-DB and IMGT/DomainGapAlign: a database and a tool for immunoglobulins or antibodies, T cell receptors, MHC, IgSF and MhcSF. Nucleic Acids Res 2010; 38:D301-7.
17. Brochet X, Lefranc M P, Giudicelli V. IMGTN-QUEST: the highly customized and integrated system for IG and TR standardized V-J and V-D-J sequence analysis. Nucleic Acids Res 2008; 36:W503-8.
18. Giudicelli V, Chaume D, Lefranc MP. IMGT/GENE-DB: a comprehensive database for human and mouse immunoglobulin and T cell receptor genes. Nucleic Acids Res 2005; 33:D256-61.
19. Lefranc M P. IMGT, the international ImMunoGeneTics information system for Immunoinformatics. Methods for querying IMGT databases, tools, and Web resources in the context of immunoinformatics. Methods Mol Biol 2007; 409:19-42.
20. Kaas Q, Ehrenmann F, Lefranc MP. IG, TR and IgSF, MHC and MhcSF: what do we learn from the IMGT Colliers de Perles? Brief Funct Genomic Proteomic 2007; 6: 253-64.
21. Kaas Q, Ruiz M, Lefranc M P. IMGT/3Dstructure-DB and IMGT/StructuralQuery, a database and a tool for immunoglobulin, T cell receptor and MHC structural data. Nucleic Acids Res 2004; 32:D208-10.
22. Giuliano S, Cormerais Y, Dufies M, et al. Resistance to sunitinib in renal clear cell carcinoma results from sequestration in lysosomes and inhibition of the autophagic flux. Autophagy 2015; 11:1891-904.
23. Lupu-Plesu M, Claren A, Martial S, et al. Effects of proton versus photon irradiation on (lymph)angiogenic, inflammatory, proliferative and anti-tumor immune responses in head and neck squamous cell carcinoma. Oncogenesis 2017; 6:e354.
24. Dumond, A.; Brachet, E.; Durivault, J.; Vial, V.; Puszko, A.K.; Lepelletier, Y.; Montemagno, C.; Pagnuzzi-Boncompagni, M.; Hermine, O.; Garbay, C., et al. Neuropilin 1 and Neuropilin 2 gene invalidation or pharmacological inhibition reveals their relevance for the treatment of metastatic renal cell carcinoma. J Exp Clin Cancer Res 2021, 40, 33, doi:10.1186/s13046-021-01832-x.
25. Signoretti, S.; Flaifel, A.; Chen, Y. B.; Reuter, V. E. Renal Cell Carcinoma in the Era of Precision Medicine: From Molecular Pathology to Tissue-Based Biomarkers. J Clin Oncol 2018, 10.1200/JCO.2018.79.2259, JCO2018792259, doi:10.1200/JCO.2018.79.2259.
26. Grepin, R.; Ambrosetti, D.; Marsaud, A.; Gastaud, L.; Amiel, J.; Pedeutour, F.; Pages, G. The relevance of testing the efficacy of anti-angiogenesis treatments on cells derived from primary tumors: a new method for the personalized treatment of renal cell carcinoma. PLoS ONE 2014, 9, e89449.

1-15. (canceled)

16. An isolated anti-vascular endothelial growth factor-C (VEGFC) antibody or a functional fragment thereof, said antibody comprising a heavy chain comprising at least one of CDR-H1, CDR-H2 and CDR-H3 of amino acid sequences SEQ ID N° 6, 7 and 8 and a light chain comprising at least one of CDR-L1, CDR-L2 and CDR-L3 of amino acid sequences SEQ ID N° 9, 10 and 11, respectively.

17. The antibody of claim 16, wherein the heavy chain variable region (VH) comprises:
a heavy chain CDR-H1 of sequence SEQ ID NO: 6; and
a heavy chain CDR-H2 of sequence SEQ ID NO: 7; and
a heavy chain CDR-H3 of sequence SEQ ID NO: 8
and wherein the light chain variable region (V_{L}) comprises:
a light chain CDR-L1 of sequence SEQ ID NO: 9; and
a light chain CDR-L2 of sequence SEQ ID NO: 10 and
a light chain CDR-L3 of sequence SEQ ID NO:11.

18. The antibody according to claim 16, wherein the sequence of the heavy chain variable region (VH) comprises or consists of the sequence SEQ ID NO: 4; and
wherein the sequence of the light chain variable region (VL) comprises or consists of the sequence SEQ ID NO: 5.

19. The antibody according to claim 16, wherein said antibody is a monoclonal antibody.

20. The antibody according to claim 16, wherein said antibody is a chimeric antibody or a humanized antibody.

21. A polynucleotide encoding a variable heavy chain (V_{H}) or a variable light chain (VL) for an anti-VEGFC antibody as defined in claim 16.

22. An expression vector comprising a polynucleotide as defined in claim 21.

23. A method of producing an anti-VEGFC antibody according to claim 16 comprising:
culturing the non-human host cell transformed with pairs of polynucleotides suitable for expressing an anti-VEGFC according to claim 16, under suitable conditions and
recovering the anti-VEGFC antibody from the culture medium or from the said cultured cells.

24. A combination of an anti-VEGFC antibody according to claim 16 and an anti-angiogenic compound.

25. The combination of claim 24, said anti-VEGFC antibody comprising a heavy chain comprising at least one of CDR-H1, CDR-H2 and CDR-H3 of amino acid sequences SEQ ID N° 6, 7 and 8 and a light chain comprising at least one of CDR-L1, CDR-L2 and CDR-L3 of amino acid sequences SEQ ID N° 9, 10 and 11, respectively, wherein the anti-angiogenic compound is selected from the group consisting of anti-VEGF antibodies distinct from said anti-VEGFC antibody, inhibitors of receptors involved in angiogenesis including VEGFR1, 2, 3, CSFR, PDGFR, inhibitors of m-TOR.

26. The combination according to claim 25, wherein the anti-angiogenic compound is selected from the group consisting of bevacizumab and aflibercept.

27. A pharmaceutical composition comprising an antibody according to claim 16 and an excipient, carrier, and/or diluent.

28. A method of treatment of cancers or disorders characterized by undesirable lymphatic endothelial cell migration and/or proliferation, comprising the administration into a subject in need thereof of an antibody as defined in claim 16.

29. A method according to claim 28 for the treatment renal cancer carcinoma (RCC), breast cancer, head and neck cancers, retinopathies, arthritis or rheumatoid arthritis.

30. A method according to claim 29, wherein the subject is relapsed from or refractory to its conventional treatment, and/or is developing or is at risk of developing tumor metastasis.

31. An in vitro method for diagnosing or predicting the risk to relapse and/or to develop a tumor metastasis in a subject treated by its conventional treatment comprising an anti-angiogenic compound, wherein said method comprises a step of contacting a biological sample of said subject with a labelled or non-labelled anti-VEGFC antibody of claim 16.

32. A kit comprising at least one anti-VEGFC antibody and/or fragment thereof as defined in claim 16 and at least one reagent for detecting said anti-VEGFC antibody.
